SEQUENCE LISTING (24 sequences)

SEQ ID NO: 1 - protein, 117 aa, Artificial Sequence
  Synthetic Construct
QLLFNKTKSVEFTFSNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVS

SEQ ID NO: 2 - protein, 120 aa, Artificial Sequence
  Synthetic Construct
WQLPLLFNKTKSVEFTFGNDTVVIPCFVTNMEAQNTTEVYVKWKFKGRDIYTFDGDKNKSTVPTDFSSAKIEVSQLLKGDASLKMDKSDAVSHTGNYTCEVTELTREGETIIELKYRVVS

SEQ ID NO: 3 - protein, 120 aa, Artificial Sequence
  Synthetic Construct
WQPPLLFNKTKSVEFTFGNDTVVIPCFVTNMEAQNTTEVYVKWKFKGRDIYTFDGQANKSTVPTDFSSAKIEVSQLLKGDASLKMDKSDAVSHTGNYTCEVTELTREGETIIELKYRVVS

SEQ ID NO: 4 - protein, 120 aa, Artificial Sequence
  Synthetic Construct
WQPPLLFNKTKSVEFTFCNDTVVIPCFVTNMEAQNTTEVYVKWKFKGRDIYTFDGQANKSTVPTDFSSAKIEVSQLLKGDASLKMDKSDAVSHTGNYTCEVTELTREGETIIELKYRVVS

SEQ ID NO: 5 - protein, 120 aa, Artificial Sequence
  Synthetic Construct
WQPPLLFNKTKSVEFTCGNDTVVIPCFVTNMEAQNTTEVYVKWKFKGRDIYTFDGQANKSTVPTDFSSAKIEVSQLLKGDASLKMDKSDAVSHTGNYTCEVTELTREGETIIELKYRVVS

SEQ ID NO: 6 - protein, 119 aa, Artificial Sequence
  Synthetic Construct
DVQLVESGGGVVRPGESLRLSCAASGFSFSSYAMNWVRQAPGEGLEWVSRINSGGGGTDYAESVKGRFTISRDNSENTLYLQMNSLRAEDTAVYYCAKQYDWNSFFDYWGLGALVTVSS

SEQ ID NO: 7 - protein, 108 aa, Artificial Sequence
  Synthetic Construct
ETVLTQSPATLSVSPGERATLSCRASQTVGSKLAWHQQKPGQAPRLLIYDATNRATGISDRFSGSGSGTDFTLTISSLQTEDSAVYYCQQYYYWPPYRFGGGTKVEIK

SEQ ID NO: 8 - protein, 119 aa, Artificial Sequence
  Synthetic Construct
EEELQIIQPDKSVLVAAGETATLRCTITSLFPVGPIQWFRGAGPGRVLIYNQRQGPFPRVTTVSDTTKRNNMDFSIRIGAITPADAGTYYCIKFRKGSPDDVEFKSGAGTELSVRAKPS

SEQ ID NO: 9 - protein, 119 aa, Artificial Sequence
  Synthetic Construct
EEELQIIQPDKSVLVAAGETATLRCTITSLFPVGPIQWFRGAGPGRELIYNQREGPFPRVTTVSDTTKRNNMDFSIRIGAITPADAGTYYCVKFRKGSPDDVEFKSGAGTELSVRAKPS

SEQ ID NO: 10 - protein, 119 aa, Artificial Sequence
  Synthetic Construct
EEELQIIQPDKSVLVAAGETATLRCTITSLFPVGPIQWFRGAGPGRVLIYNQREGPFPRVTTVSDTTKRNNMDFSIRIGAITPADAGTYYCIKFRKGSPDDVEFKSGAGTELSVRAKPS

SEQ ID NO: 11 - protein, 119 aa, Artificial Sequence
  Synthetic Construct
EDELQIIQPEKSVSVAAGESATLRCAITSLFPVGPIQWFRGAGAGRVLIYNQRQGPFPRVTTVSETTKRNNLDFSISISNITPADAGTYYCIKFRKGSPDDVEFKSGAGTELSVRAKPS

SEQ ID NO: 12 - protein, 119 aa, Artificial Sequence
  Synthetic Construct
EEELQIIQPDKSISVAAGESATLHCTITSLFPVGPIQWFRGAGPGRVLIYNQRQGPFPRVTTVSDTTKRNNMDFSIRISNITPADAGTYYCIKFRKGSPDDVEFKSGAGTELSVRAKPS

SEQ ID NO: 13 - protein, 119 aa, Artificial Sequence
  Synthetic Construct
EEELQIIQPEKLLLVTVGKTATLHCTITSLFPVGPIQWFRGVGPGRVLIYNQRDGPFPRVTTVSDGTKRNNMDFSIRISSITPADVGTYYCVKFRKGTPEDVEFKSGPGTEMALGAKPS

SEQ ID NO: 14 - protein, 119 aa, Artificial Sequence
  Synthetic Construct
EEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPS

SEQ ID NO: 15 - protein, 119 aa, Artificial Sequence
  Synthetic Construct
EEELQIIQPEKLLLVTVGKTATLHCTITSLFPVGPIQWFRGVGPGRVLIYNQKDGHFPRVTTVSDGTKRNNMDFSIRISSITPADVGTYYCVKFRKGSPEDVEFKSGPGTEMALGAKPS

SEQ ID NO: 16 - protein, 119 aa, Artificial Sequence
  Synthetic Construct
EEELQIIQPEKLLLVTVGKTATLHCTITSHFPVGPIQWFRGVGPGRVLIYNQKDGHFPRVTTVSDGTKRNNMDFSIRISSITPADVGTYYCVKFRKGSPEDVEFKSGPGTEMALGAKPS

SEQ ID NO: 17 - protein, 119 aa, Artificial Sequence
  Synthetic Construct
EEELQIIQPEKLLLVTVGKTATLHCTITSLFPVGPVLWFRGVGPGRVLIYNQRQGPFPRVTTVSDTTKRNNMDFSIRISSITPADVGTYYCVKFRKGTPEDVEFKSGPGTEMALGAKPS

SEQ ID NO: 18 - protein, 119 aa, Artificial Sequence
  Synthetic Construct
EEELQIIQPEKLLLVTVGKTATLHCTITSLFPVGPIQWFRGVGPGRELIYNAREGRFPRVTTVSDLTKRNNMDFSIRISSITPADVGTYYCVKFRKGSPEDVEFKSGPGTEMALGAKPS

SEQ ID NO: 19 - protein, 119 aa, Artificial Sequence
  Synthetic Construct
EEELQIIQPEKLLLVTVGKTATLHCTITSLLPVGPIQWFRGVGPGRELIYNQRDGPFPRVTTVSDGTKRNNMDFSIRISSITPADVGTYYCVKFRKGTPEDVEFKSGPGTEMALGAKPS

SEQ ID NO: 20 - protein, 119 aa, Artificial Sequence
  Synthetic Construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPS

SEQ ID NO: 21 - protein, 117 aa, Artificial Sequence
  Synthetic Construct
DVQLVESGGGVVRPGESLRLSCEASGFTFSSNAMSWVRQAPGKGLEWVAGISSGSDTYYGDSVKGRLTISRDNSKNILYLQMNSLTAEDTAVYYCARETWNHLFDYWGQGTLVTVSS

SEQ ID NO: 22 - protein, 108 aa, Artificial Sequence
  Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTKLTVL

SEQ ID NO: 23 - protein, 119 aa, Artificial Sequence
  Synthetic Construct
DVQLVESGGGVVRPGESLRLSCAASGFTFSSYDMNWVRQAPGEGLEWVSLISGSGEIIYYADSVKGRFTISRDNSKNTLYLQMNSLRAEDTAVYYCAKENNRYRFFDDWGQGTLVTVSS

SEQ ID NO: 24 - protein, 108 aa, Artificial Sequence
  Synthetic Construct
ETVLTQSPGTLTLSPGERATLTCRASQSVYTYLAWYQEKPGQAPRLLIYGASSRATGIPDRFSGSGSGTEFTLTISSLQSEDFAVYYCQQYYDRPPLTFGGGTKVEIK

(19) United States
(12) Patent Application Publication
(10) Pub. No.: US 2020/0400662 A1
(43) Pub. Date: Dec. 24, 2020

(54) METHODS AND REAGENTS FOR REDUCING THE INTERFERENCE OF DRUGS THAT BIND CD47 IN SEROLOGICAL ASSAYS

(71) Applicant: ALX ONCOLOGY INC., Burlingame, CA (US)

(72) Inventors: Hong WAN, Foster City, CA (US); Jaume Pons, San Francisco, CA (US); Sophia Randolph, Chico, CA (US); Bang Janet Sim, Brisbane, CA (US)

(21) Appl. No.: 16/894,468

(22) Filed: Jun. 5, 2020

Related U.S. Application Data
(60) Provisional application No. 62/934,395, filed on Nov. 12, 2019.
(60) Provisional application No. 62/858,871, filed on Jun. 7, 2019.

(51) Int. Cl. G01N 33/543; C07K 16/28; G01N 33/68

(52) U.S. Cl. CPC G01N 33/54393; C07K 16/2803; G01N 33/68; C07K 2317/52; G01N 2440/34; C07K 2317/54; C07K 2317/55; C07K 2317/622; C07K 2317/626; C07K 2317/56

(57) ABSTRACT

Provided are methods of reducing and/or preventing interference by a drug comprising (i) an antibody Fc region and (ii) a moiety that binds to human CD47 in serological assays.

### CROSS REFERENCE TO RELATED APPLICATIONS

This application claims the priority benefit of U.S. Provisional Application No. 62/858,871, filed Jun. 7, 2019, and U.S. Provisional Application No. 62/934,395, filed Nov. 12, 2019, the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 757972000900SEQLIST.TXT, date recorded: Jun. 3, 2020, size: 26 KB).

### FIELD OF THE INVENTION

This invention relates to methods and reagents for use in reducing interference in serological assays by drugs that comprise (i) an antibody Fc region and (ii) a moiety that binds to human CD47.

### BACKGROUND OF THE INVENTION

An ever-increasing number of antibody-based drugs are being developed as treatments for a wide variety of diseases, including cancer. Such treatments have the potential of interfering with blood typing and serological assays if the target of the therapeutic antibody is also expressed on blood cells such as red blood cells (RBCs), white blood cells (WBC) and/or platelets.

For example, CD47, a widely-expressed cell surface protein that binds to signal regulatory protein-α (SIRPα) and inhibits phagocytosis (Jaiswal et al., Trends Immunol (2010) 31(6):212-219; Brown et al., Trends Cell Biol (2001) 11(3):130-135), is expressed at high levels on a wide variety of malignant tumors, including hematological and solid tumors. Elevated CD47 expression also correlates with aggressive disease (Willingham et al., Proc Nat Acad Sci USA (2012) 109(17):6662-6667). Several cancer therapies targeting CD47, e.g., antibodies and fusion proteins comprising an antibody Fc region, have been developed to block the SIRPα-CD47 interaction, thereby permitting macrophages to carry out their phagocytic function to clear tumor cells.

As CD47 is also expressed on the surface of blood cells, such as red blood cells (RBCs) and platelets (Oldenborg et al., Science (2000) 288(5473):2051-2054), drugs comprising antibody Fc regions that target CD47 could interfere with blood typing and serological tests. Moreover, because patients receiving CD47-targeting drugs (e.g., for the treatment of cancer) often require blood transfusions to treat coincident anemia and/or thrombocytopenia, interference with serological and blood typing assays by anti-CD47 drugs is a significant patient safety concern. Thus, there is need in the art to develop methods and reagents to reduce interference of CD47-targeting drugs comprising antibody Fc regions with serological assays.

### SUMMARY OF THE INVENTION

Provided is a method of reducing drug interference in a serological assay using reagent red blood cells (RBC) or reagent platelets, said method comprising: (a) adding a drug neutralizing agent that binds to a drug and blocks the drug from binding the reagent RBC or the reagent platelets to a plasma sample from a subject who has received treatment with the drug; and (b) performing the serological assay of the plasma sample after step (a) using the reagent RBC or the reagent platelets, wherein the drug comprises (i) a human antibody Fc region or variant thereof and (ii) a moiety that binds to human CD47.

In some embodiments, the moiety of the drug that binds to human CD47 comprises a wild type SIRPα, a SIRPα variant, or a fragment of the wild type SIRPα or the SIRPα variant. In some embodiments, the moiety of the drug that binds to human CD47 comprises the SIRPα variant, and wherein the SIRPα variant comprises one or more amino acid substitution(s), insertion(s), deletion(s), N-terminal extension(s), and/or C-terminal extension(s) relative to the wild type SIRPα. In some embodiments, the moiety of the drug that binds to human CD47 comprises the fragment of the SIRPα variant, and wherein the fragment comprises an extracellular domain of the SIRPα variant. In some embodiments, the drug neutralizing agent is an anti-SIRPα antibody that is capable of binding the wild type SIRPα, the SIRPα variant, or the fragment of the wild type SIRPα or the SIRPα variant.

In some embodiments, the moiety of the drug that binds to human CD47 comprises a wild type SIRPγ, a SIRPγ variant, or a fragment of the wild type SIRPγ or the SIRPγ variant. In some embodiments, the moiety of the drug that binds to human CD47 comprises the SIRPγ variant, and wherein the SIRPγ variant comprises one or more amino acid substitution(s), insertion(s), deletion(s), N-terminal extension(s), C-terminal extension(s), or any combination of the preceding, relative to the wild type SIRPγ. In some embodiments, the moiety of the drug that binds to human CD47 comprises the fragment of the SIRPγ variant, and wherein the fragment comprises an extracellular domain of the SIRPγ variant. In some embodiments, the drug neutralizing agent is an anti-SIRPγ antibody that is capable of binding the wild type SIRPγ, the SIRPγ variant, or the fragment of the wild type SIRPγ or the SIRPγ variant.

In some embodiments, the moiety of the drug that binds to human CD47 comprises a SIRPγ variant or a fragment of the SIRPγ variant. In some embodiments, the moiety of the drug that binds to human CD47 comprises the SIRPγ variant, and wherein the SIRPγ variant comprises one or more amino acid substitution(s), insertion(s), deletion(s), N-terminal extension(s), C-terminal extension(s), or any combination of the preceding, relative to the wild type SIRPγ. In some embodiments, the moiety of the drug that binds to human CD47 comprises the fragment of the SIRPγ variant, and wherein the fragment comprises an extracellular domain of the SIRPγ variant. In some embodiments, the drug neutralizing agent is an anti-SIRPβ antibody that is capable of binding the SIRPγ variant or the fragment of the SIRPγ variant.

In some embodiments, the drug is an anti-CD47 antibody. In some embodiments, the drug neutralizing agent is an anti-idiotypic antibody that binds the antigen binding portion of the anti-CD47 antibody.

In some embodiments, the drug neutralizing agent is a CD47 polypeptide capable of binding the moiety of the drug that binds to human CD47. In some embodiments, the CD47 polypeptide is a monomer, a dimer, or an oligomer. In some embodiments, the CD47 polypeptide is a human CD47, a mouse CD47, a rat CD47, a rhesus CD47, or a cynomolgus CD47. In some embodiments, the CD47 polypeptide comprises the amino acid sequence of SEQ ID NO: 1. In some embodiments, the CD47 polypeptide is a CD47 variant that comprises one or more amino acid substitutions, insertions, deletions, N-terminal extensions, or C-terminal extensions relative to the wildtype CD47. In some embodiments, the CD47 variant comprises the amino acid sequence set forth in any one of SEQ ID NOs: 2-5.

In some embodiments, the affinity of the drug neutralizing agent for the drug is higher than the affinity of the drug for human CD47. In some embodiments, the drug neutralizing agent is added to the plasma sample in a molar excess amount relative to the amount of drug in the plasma sample.

Also provided is a method of reducing drug interference in a serological assay of a blood sample containing red blood cells (RBC) and/or platelets, said method comprising: (a) adding an anti-SIRPα antibody to the blood sample from a subject who has received treatment with a drug; and (b) performing the serological assay of the blood sample after step (a), wherein the drug comprises (i) an antibody Fc region and (ii) an extracellular domain of a wild type SIRPα or a variant thereof that binds to human CD47, and wherein the anti-SIRPα antibody fragment displaces the drug bound to CD47 on the surface of the RBC in the blood sample. In some embodiments, the anti-SIRPα antibody comprises (a) a heavy chain variable domain (V_{H}) that comprises SEQ ID NO: 6 and a light chain variable domain (V_{L}) that comprises SEQ ID NO: 7; (b) a heavy chain variable domain (V_{H}) that comprises SEQ ID NO: 21 and a light chain variable domain (V_{L}) that comprises SEQ ID NO: 22; or (c) a heavy chain variable domain (V_{H}) that comprises SEQ ID NO: 23 and a light chain variable domain (V_{L}) that comprises SEQ ID NO: 24. In some embodiments, the drug comprises a variant of an extracellular domain of the wild type SIRPα. In some embodiments, the variant comprises one or more amino acid substitution(s), insertion(s), deletion(s), N-terminal extension(s), and/or C-terminal extension(s) relative to the extracellular domain the wild type SIRPα.

In some embodiments, the anti-SIRPα antibody is added to the blood sample in a molar excess amount relative to the amount of drug in the blood sample.

Also provided is a method of reducing drug interference in a serological assay using reagent red blood cells (RBCs), reagent platelets, or a combination thereof said method comprising: (a) adding a cell binding agent to the reagent red blood cells (RBCs), reagent platelets, or combination thereof, wherein the cell binding agent binds to human CD47 and does not comprise an antibody Fc region; (b) performing the serological assay of a plasma sample using the reagent red blood cells (RBCs), reagent platelets, or combination thereof of step (a), wherein the plasma sample is from a subject who has received treatment with a drug, and wherein the drug comprises (i) an antibody Fc region and (ii) a moiety that binds to human CD47.

In addition, provided is a method of reducing drug interference in a serological assay using reagent red blood cells (RBCs), reagent platelets, or a combination thereof, said method comprising: (a) adding a cell binding agent a plasma sample from a subject who has received treatment with a drug, wherein the cell binding agent binds to human CD47 and does not comprise an antibody Fc region; and (b) performing the serological assay of the plasma sample after step (a) using the reagent red blood cells (RBCs), reagent platelets, or combination thereof, wherein the drug comprises (i) an antibody Fc region and (ii) a moiety that binds to human CD47.

Also provided is a method of reducing drug interference in a serological assay of a blood sample containing reagent red blood cells (RBCs), reagent platelets, or a combination thereof, said method comprising: (a) adding a cell binding agent that binds to human CD47 and does not comprise an antibody Fc region to a blood sample from a subject who has received treatment with a drug; and (b) performing the serological assay of the blood sample after step (a), wherein the drug comprises (i) an antibody Fc region and (ii) a moiety that binds to human CD47.

In some embodiments, the cell binding agent comprises a wild type SIRPα, wild type SIRPγ, or a fragment of the wild type SIRPα or the wild type SIRPγ that is capable of binding human CD47. In some embodiments, the cell binding agent comprises a SIRPα variant that is capable of binding human CD47, or a CD47-binding fragment thereof. In some embodiments, the SIRPα variant comprises one or more amino acid substitution(s), insertion(s), deletion(s), N-terminal extension(s), C-terminal extension(s), or any combination of the preceding, relative to the wild type SIRPα. In some embodiments, the cell binding agent comprises a SIRPγ variant that is capable of binding human CD47, or a CD47-binding fragment thereof. In some embodiments, the SIRPγ variant comprises one or more amino acid substitution(s), insertion(s), deletion(s), N-terminal extension(s), C-terminal extension(s), or any combination of the preceding, relative to the wild type SIRPγ. In some embodiments, the cell binding agent comprises a SIRPγ variant that is capable of binding human CD47, or a CD47-binding fragment thereof. In some embodiments, the SIRPγ variant comprises one or more amino acid substitution(s), insertion(s), deletion(s), N-terminal extension(s), C-terminal extension(s), or any combination of the preceding, relative to the wild type SIRPγ. In some embodiments, the variant comprises the amino acid sequence of any one of SEQ ID NOs: 8-16.

In some embodiments, the cell binding agent comprises an antigen-binding fragment of an anti-CD47 antibody. In some embodiments, the antigen binding fragment is a Fab, a Fab′, a Fab′-SH, an F(ab′)2, an Fv, an scFv, or a diabody.

In some embodiments, the affinity of the cell binding agent for human CD47 is higher than the affinity of the drug for human CD47. In some embodiments, the cell binding agent is added to the reagent RBC and/or reagent platelets in a molar excess amount relative to the amount of drug in the plasma sample. In some embodiments, the cell binding agent is added to the plasma sample in a molar excess amount relative to the amount of drug in the plasma sample. In some embodiments, the SIRPα agent is added to the blood sample in a molar excess amount relative to the amount of drug in the blood sample.

In some embodiments, the antibody Fc region of the drug is a human IgG Fc region or a variant thereof. In some embodiments, the human IgG Fc region is an IgG1, IgG2, or IgG4 Fc region, or a variant of an IgG1, IgG2, or IgG4 Fc region.

In some embodiments, the serological assay is an ABO/Rh typing assay. In some embodiments, the serological assay is an immediate spin (IS) assay. In some embodiments, the serological assay is a direct antiglobulin (DAT) assay using a polyspecific reagent that detects IgG and complement C3. In some embodiments, the serological assay is a direct antiglobulin (DAT) assay using a monospecific reagent that detects complement C3. In some embodiments, the serological assay is a PEG-enhanced serological assay. In some embodiments, the serological assay is an eluate test that is performed following the DAT assay.

Also provided is polypeptide comprising any one of SEQ ID NOs: 11-20.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF DESCRIPTION OF THE DRAWINGS

FIG. 1A shows a serological assay in which a plasma sample obtained from a subject is mixed with reagent red blood cells (i.e., red blood cells (“RBCs”) that are known to express a particular cell surface antigen, or group of cell surface antigens) to detect the presence of antibodies in the plasma sample that bind the RBC surface antigen. Alternatively, such serological assay can be performed using reagent platelets (i.e., platelets that are known to express a particular cell surface antigen, or group of cell surface antigens) instead of reagent RBCs.

FIG. 1B shows how the presence of a drug comprising (i) and antibody Fc region and (ii) a moiety that binds human CD47 in a plasma sample interferes with the assay of FIG. 1A.

FIG. 1C shows a serological assay in which a blood sample obtained from a subject is mixed with reagent plasma/antisera (i.e., plasma or antisera known to contain antibodies against a specific RBC surface antigen(s) or platelet surface antigen(s)) in order to detect the presence of the antigen on the subject's RBCs and/or platelets.

FIG. 1D shows how the presence of a drug comprising (i) and antibody Fc region and (ii) a moiety that binds human CD47 in a blood sample interferes with the assay of FIG. 1C.

FIG. 2 shows a method of reducing interference in a serological assay that comprises adding a drug neutralizing agent to a plasma sample obtained from a subject who has been treated with the drug. Briefly, the drug neutralizing agent binds the drug in the plasma sample so that little or no free drug is available to bind the CD47 on the surface of the reagent RBCs or reagent platelets.

FIG. 3A shows a method of reducing interference in a serological assay that comprises adding an anti-SIRPα antibody to a plasma sample from a subject who has received treatment with the drug. The anti-SIRPα antibody binds the drug in the plasma sample so that little or no free drug is available to bind the CD47 on the surface of the reagent RBCs or reagent platelets.

FIG. 3B shows a method of reducing interference in a serological assay that comprises adding an anti-SIRPα antibody to a blood sample from a subject who has received treatment with the drug. The anti-SIRPα antibody displaces the drug bound to the CD47 on the surface of the subject's RBCs and/or platelets and minimizes the amount of drug-bound RBC and/or drug-bound platelets in the patient's blood sample (or eliminates drug-bound RBC and/or drug-bound platelets in the sample).

FIG. 4A shows a method of reducing interference in a serological assay that comprises adding a cell binding agent to reagent RBCs or reagent platelets. Briefly, a cell binding agent that binds human CD47 and does not comprise an antibody Fc region binds to the CD47 on the surface of the reagent RBCs or reagent platelets. Binding of the reagent RBCs (or reagent platelets) by the cell binding agent blocks the drug from binding the reagent RBCs (or reagent platelets).

FIG. 4B shows a method of reducing interference in a serological assay that comprises adding a cell binding agent to plasma from a subject who has received treatment with the drug. Briefly, the cell binding agent competes with the drug for binding to the CD47 expressed on the surface of the reagent RBCs or reagent platelets and minimizes the amount of drug-bound reagent RBCs or drug-bound reagent platelets in the assay (or eliminates drug-bound reagent RBC and/or drug-bound reagent platelets in the assay).

FIG. 4C shows a method of reducing interference in a serological assay that comprises adding a cell binding agent to a blood sample from a subject who has received treatment with the drug. Briefly, the cell binding agent competes with the drug for binding to the CD47 expressed on the surface of the subject's RBCs and/or platelets and minimizes (or eliminates) the amount of drug-bound RBC and/or drug-bound platelets in the assay.

FIG. 5A shows the results of hemagglutination experiments that were performed to determine whether Antibody A, an exemplary anti-SIRPα antibody that blocks the interaction between SIRPα and CD47, mitigates the interference of Drug A bound to CD47 on the surface of erythrocytes.

FIG. 5B shows the results of experiments that were performed to determine whether Antibody A displaces Drug A bound to CD47 on the surface of DLD-1 cells.

### DETAILED DESCRIPTION OF THE INVENTION

### Methods of Mitigating Interference in Pre-Transfusion Serological Assays

CD47 is a transmembrane protein that interacts with thrombospondin-1 (TSP-1) as well as several molecules on immune cells, including signal regulatory protein alpha (SIRPα). Upon binding CD47, SIRPα initiates a signaling cascade that inhibits phagocytosis and prevents phagocytic removal of healthy cells by the immune system. However, many cancers overexpress CD47 and evade phagocytic clearance. Accordingly, drugs that target CD47 (such as anti-CD47 antibodies and fusion proteins comprising an antibody Fc region and a moiety that binds CD47) are of significant therapeutic interest. CD47 is also expressed on the surface of human red blood cells (RBCs) and platelets. Thus, following the administration of a drug comprising (i) an antibody Fc region and (ii) a moiety that binds to human CD47 to a subject, the drug present in the subject's plasma or bound to the subject's RBCs and/or platelets may cause interference in routine pre-transfusion serological assays.

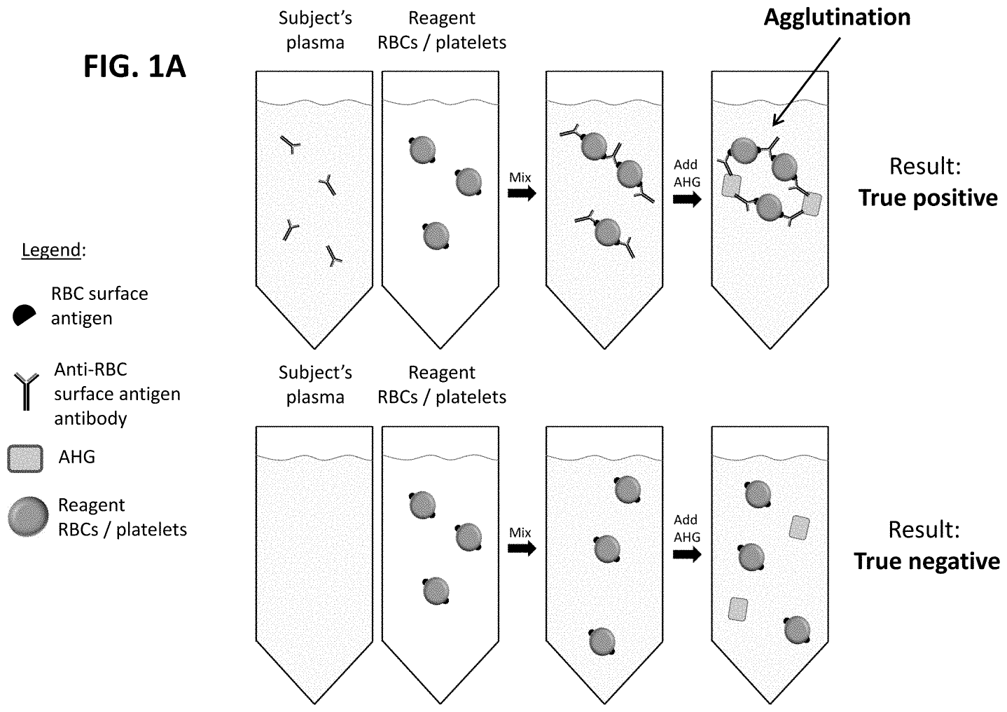

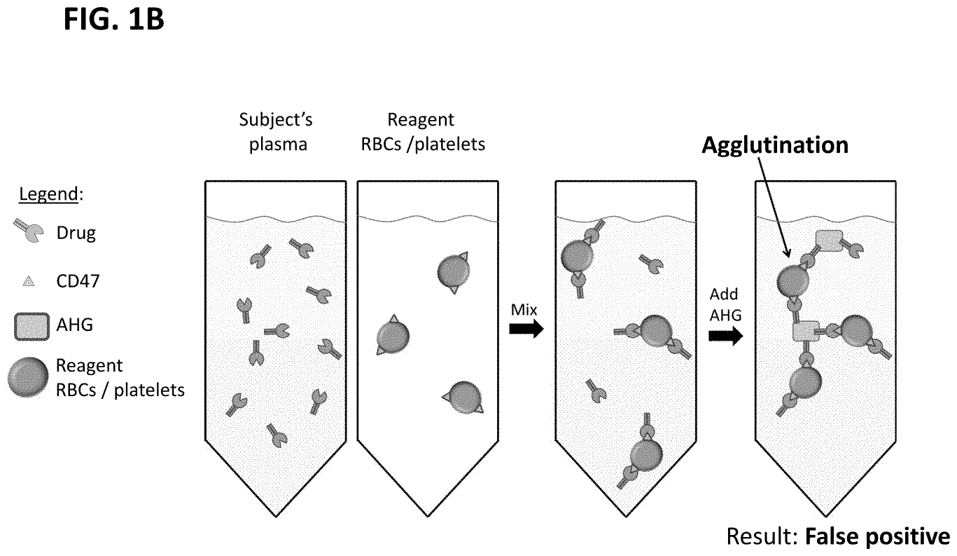

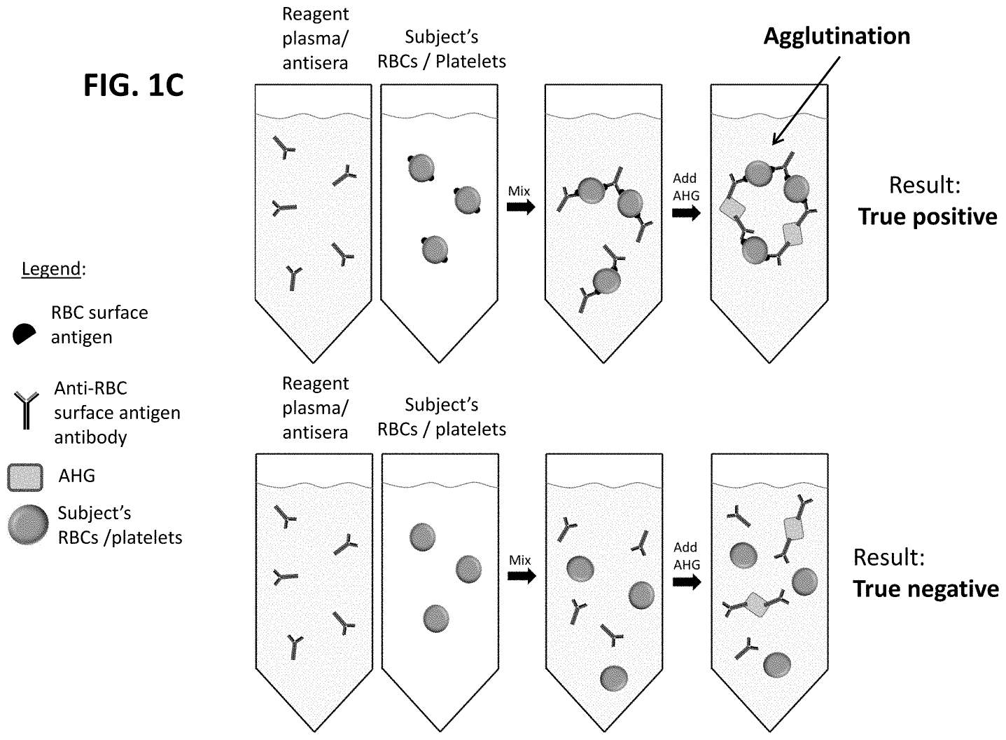

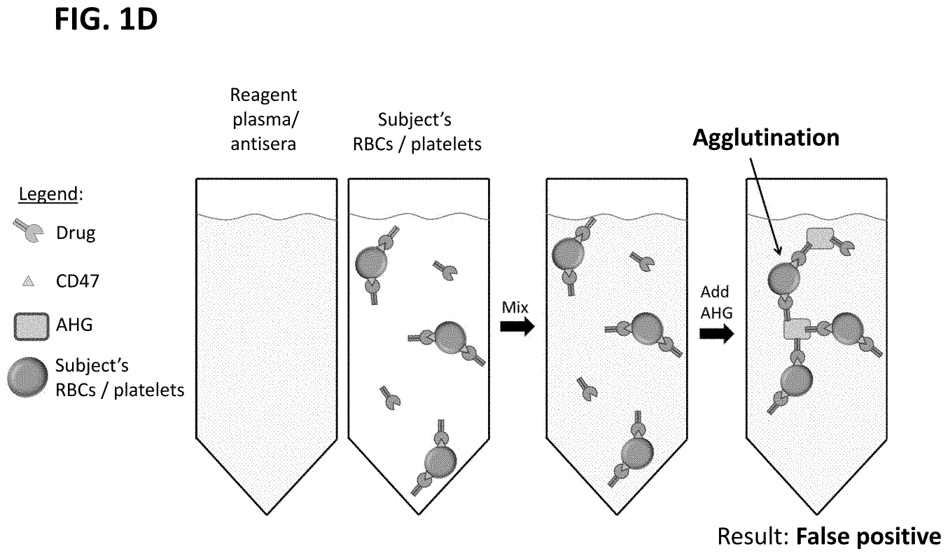

For example, FIG. 1A shows a serological assay in which a plasma sample obtained from a subject is mixed with reagent RBC or “reference RBC” (i.e., RBC that are known to express a particular cell surface antigen, or group of cell surface antigens) or reagent platelets or “reference platelets” (i.e., platelets that are known to express a particular cell surface antigen, or group of cell surface antigens) to detect the presence of antibodies in the plasma sample that bind the cell surface antigen that is known to be expressed on the reagent RBCs or reagent platelets. After the plasma sample and the reagent RBC (or reagent platelets) are mixed, anti-human globulin (AHG) is added, and agglutination (e.g., clumping) of the reagent RBC (or reagent platelets) occurs if the plasma sample contains an antibody that binds the RBC surface antigen (or platelet surface antigen). However, the presence of a drug comprising (i) an antibody Fc region and (ii) a moiety that binds to human CD47 in the subject's plasma may interfere with the assay and produce a false positive result. As shown in FIG. 1B, after the subject's plasma and the reagent RBCs (or reagent platelets) are mixed, the drug may bind CD47 that is expressed on the surface of reagent RBCs (or reagent platelets). Addition of AHG to the mix leads to agglutination of the reagent RBCs (or reagent platelets).

FIG. 1C depicts a serological assay in which a blood sample from a subject is mixed with reagent plasma/antisera (i.e., plasma or antisera containing antibodies against a known RBC surface antigen(s) or a known platelet surface antigen(s)) in order to detect the presence of the antigen on the subject's RBCs and/or platelets. After the reagent plasma/antisera and the sample from subject are mixed, the addition of AHG will lead to agglutination if the antigen recognized by the antibodies in the reagent plasma/antisera is expressed on the subject's RBCs and/or platelets. The presence of a drug comprising (i) an antibody Fc region and (ii) a moiety that binds to human CD47 in a sample comprising the subject's RBCs and/or platelets may interfere with the assay and produce a false positive result. As shown in FIG. 1D, the drug bound to the CD47 on the subject's RBCs or platelets will cause agglutination after AHG is added to a mixture comprising the subject's blood sample and reagent plasma/antisera.

The methods described below reduce (and, in some embodiments, eliminate) the interference caused by the drug, i.e., as illustrated in FIGS. 1B and 1D.

### A. Methods of Using Drug Neutralizing Agents to Mitigate Interference in a Pre-Transfusion Serological Assay

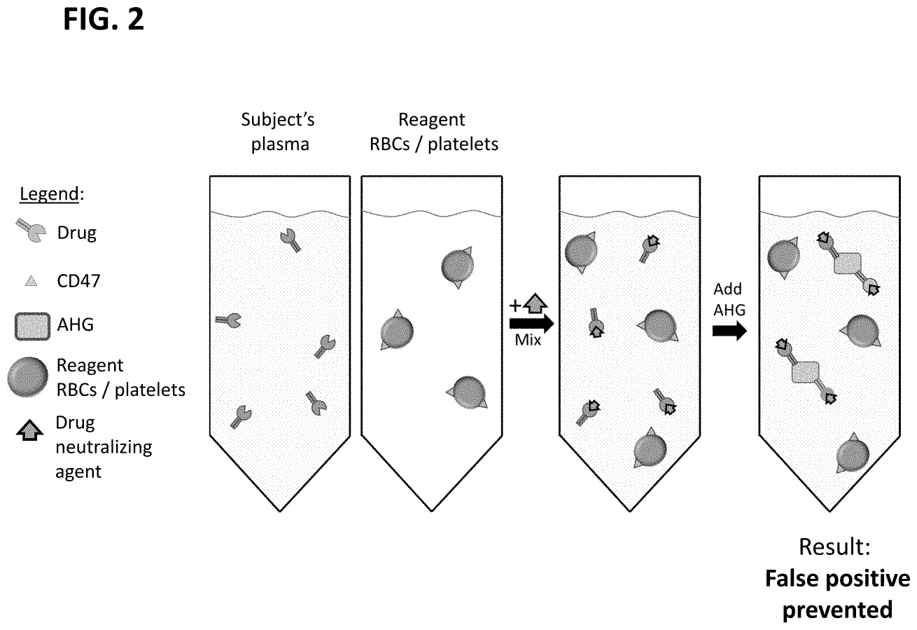

In some embodiments, the method comprises (a) adding a drug neutralizing agent that binds a drug (i.e., to the portion of the drug that comprises a moiety that binds to human CD47) to a plasma sample from a subject who has received treatment with the drug, and (b) performing the serological assay of the plasma sample after step (a) using reagent RBCs (i.e., RBCs that are known to express a particular cell surface antigen, or group of cell surface antigens) and/or reagent platelets (i.e., platelets that are known to express a particular cell surface antigen, or group of cell surface antigens), wherein the drug comprises (i) an antibody Fc region and (ii) a moiety that binds to human CD47. Such embodiments are generically depicted in FIG. 2. As shown in FIG. 2, the drug neutralizing agent binds to the drug (e.g., to the moiety of the drug that binds to human CD47) in the subject's plasma sample and blocks the drug from binding the reagent RBCs and/or reagent platelets. Little or no free drug available to bind to CD47 on the surface of the reagent RBCs and/or reagent platelets. The interference that would result from the binding of drug to the reagent RBCs and/or reagent platelets (as illustrated in FIG. 1B) is minimized (or, in some embodiments, eliminated), thus preventing a false positive result in the serological assay. In some embodiments, the drug neutralizing agent is also added to the reagent RBCs and/or reagent platelets before the serological assay is performed.

In some embodiments, the drug neutralizing agent is any agent that is capable of binding the drug and blocking the drug from binding the reagent RBCs and/or reagent platelets. In some embodiments, the drug neutralizing agent is added to the reagent RBCs and/or reagent platelets (e.g., only to the reagent RBCs and/or reagent platelets) before the serological assay is performed.

In some embodiments, the method is performed in solution, e.g., wherein the drug neutralizing agent is soluble. In some embodiments, the drug neutralizing agent is immobilized to a solid phase before the method is performed via adsorption to a matrix or surface, covalent coupling, or non-covalent coupling. In some embodiments, the drug neutralizing agent is capable of binding drug following immobilization to the solid phase. The solid phase or surface used for immobilization can be any inert support or carrier that is essentially water insoluble and useful in immunoassays, including supports in the form of, for example, surfaces, particles, porous matrices, cellulose polymer sponge (ImmunoCAP®, Phadia), and the like. Examples of commonly used supports include small sheets, Sephadex, polyvinyl chloride, plastic beads, microparticles, assay plates, or test tubes manufactured from polyethylene, polypropylene, polystyrene, and the like. In some embodiments, the drug neutralizing agent is coated on a microtiter plate, such as a multi-well microtiter plate that can be used to analyze multiple samples simultaneously.

In some embodiments, the drug comprises (i) an antibody Fc region and (ii) a SIRPα variant, a SIRPγ variant, or a SIRPγ variant that binds to human CD47, and the drug neutralizing agent comprises a CD47 polypeptide that is capable of binding to the SIRPα variant, the SIRPγ variant, or the SIRPγ variant. In some embodiments, the drug comprises an anti-CD47 antibody (e.g., an anti-human CD47 antibody), and the drug neutralizing agent comprises a CD47 polypeptide that is capable of binding to the anti-human CD47 antibody. In some embodiments, the CD47 polypeptide comprises the extracellular domain of a wild type CD47 (“WT CD47-ECD”), or a portion of WT CD47-ECD that is capable of binding the drug and blocking the drug from binding reagent RBCs and/or reagent platelets. In some embodiments, the CD47 polypeptide comprises a CD47 monomer, a CD47 dimer, or a CD47 oligomer. In some embodiments, the CD47 oligomer is an oligomer that forms spontaneously (e.g., in vitro). In some embodiments, the CD47 oligomer is an engineered oligomer, e.g., a concatenated chain of CD47 polypeptides linked via peptide bonds or linkers, a CD47 that has been engineered to comprise a domain that facilitates multimerization, or a CD47 that has been engineered to comprise tag that facilitates the binding of the CD47 to a solid support (e.g. beads, glass sides, etc.). In some embodiments, the CD47 polypeptide comprises a fusion polypeptide, e.g., a fusion polypeptide that comprises a CD47 (or a fragment thereof). In some embodiments, the fusion polypeptide comprises a CD47 (or a fragment thereof) and an antibody Fc region. In some embodiments, the CD47 polypeptide comprises a human CD47, a mouse CD47, a rat CD47, a rhesus CD47, a cynomolgus CD47, or a CD47 of any origin that is capable of binding to the drug and blocking the drug from binding reagent RBCs and/or reagent platelets. In some embodiments, the CD47 polypeptide comprises a fragment of a human CD47, mouse CD47, rat CD47, rhesus CD47, cynomolgus CD47, or CD47 of any origin, provided that the fragment is capable of binding to the drug and blocking the drug from binding reagent RBCs and/or reagent platelets. In some embodiments, the CD47 polypeptide is a variant of a wild type CD47 (or a fragment thereof, e.g., a variant of a WT CD47-ECD), provided that the variant is capable of binding to the drug. In some embodiments, the variant (or fragment thereof) comprises one or more amino acid substitution(s), deletion(s), insertion(s), N-terminal addition(s) and/or C-terminal addition(s) relative to a wild type CD47 (e.g., a wild type human, rat, mouse, rhesus, or cynomolgus CD47). In some embodiments, the one or more amino acid substitution(s), deletion(s), insertion(s), N-terminal addition(s) and/or C-terminal addition(s) present in the variant (i.e., the “CD47 variant”) alter the glycosylation pattern of the CD47 variant relative to a wild type CD47 (e.g., a wild type human, rat, mouse, rhesus, or cynomolgus CD47). In some embodiments, the one or more amino acid substitution(s), deletion(s), insertion(s), N-terminal addition(s) and/or C-terminal addition(s) present in the CD47 variant increase the affinity of the CD47 variant for the drug relative to a wild type CD47 (e.g., a wild type human, rat, mouse, rhesus, or cynomolgus CD47).

In some embodiments, the CD47 variant comprises the amino acid sequence of any one of SEQ ID NOs: 1-5 below:

(SEQ ID NO: 1)
QLLFNKTKSV EFTFSNDTVV IPCFVTNMEA QNTTEVYVKW
KFKGRDIYTF DGALNKSTVP TDFSSAKIEV SQLLKGDASL
KMDKSDAVSH TGNYTCEVTE LTREGETIIE LKYRVVS
(SEQ ID NO: 2)
WQLPLLFNKT KSVEFTFGND TVVIPCFVTN MEAQNTTEVY
VKWKFKGRDI YTFDGDKNKS TVPTDFSSAK IEVSQLLKGD
ASLKMDKSDA VSHTGNYTCE VTELTREGET IIELKYRVVS
(SEQ ID NO: 3)
WQPPLLFNKT KSVEFTFGND TVVIPCFVTN MEAQNTTEVY
VKWKFKGRDI YTFDGQANKS TVPTDFSSAK IEVSQLLKGD
ASLKMDKSDA VSHTGNYTCE VTELTREGET IIELKYRVVS
(SEQ ID NO: 4)
WQPPLLFNKT KSVEFTFCND TVVIPCFVTN MEAQNTTEVY
VKWKFKGRDI YTFDGQANKS TVPTDFSSAK IEVSQLLKGD
ASLKMDKSDA VSHTGNYTCE VTELTREGET IIELKYRVVS
(SEQ ID NO: 5)
WQPPLLFNKT KSVEFTCGND TVVIPCFVTN MEAQNTTEVY
VKWKFKGRDI YTFDGQANKS TVPTDFSSAK IEVSQLLKGD
ASLKMDKSDA VSHTGNYTCE VTELTREGET IIELKYRVVS

Additional details regarding exemplary CD47 variants that can be used as drug neutralizing agents in the methods described herein are provided in Ho et al. (2015) “‘Velcro’ Engineering of High Affinity CD47 Ectodomain as Signal Regulatory Protein a (SIRPα) Antagonists That Enhance Antibody-Dependent Cellular Phagocytosis.” J Biol Chem. 290: 12650-12663 and WO 2016/179399, the contents of which are incorporated herein by reference in their entireties.

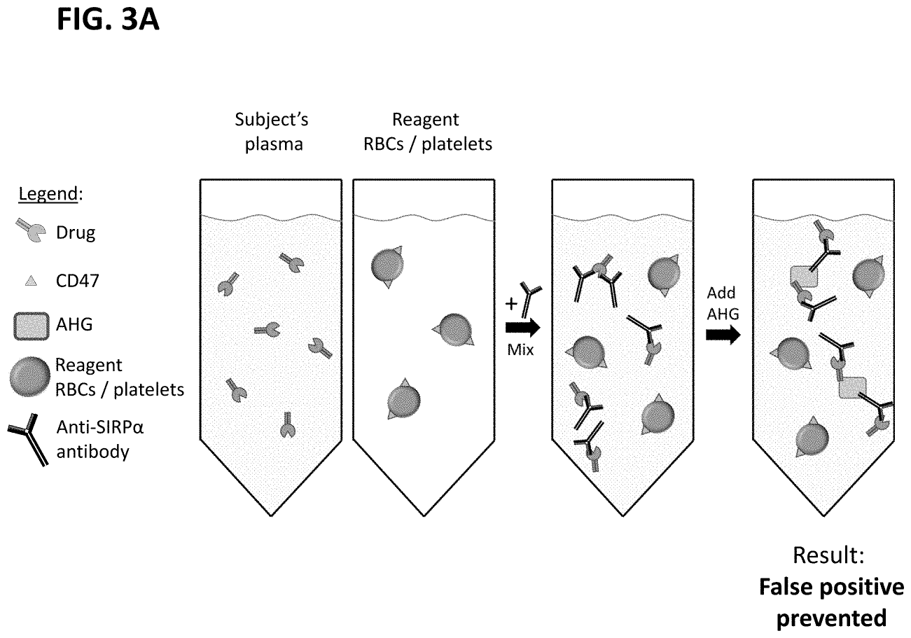

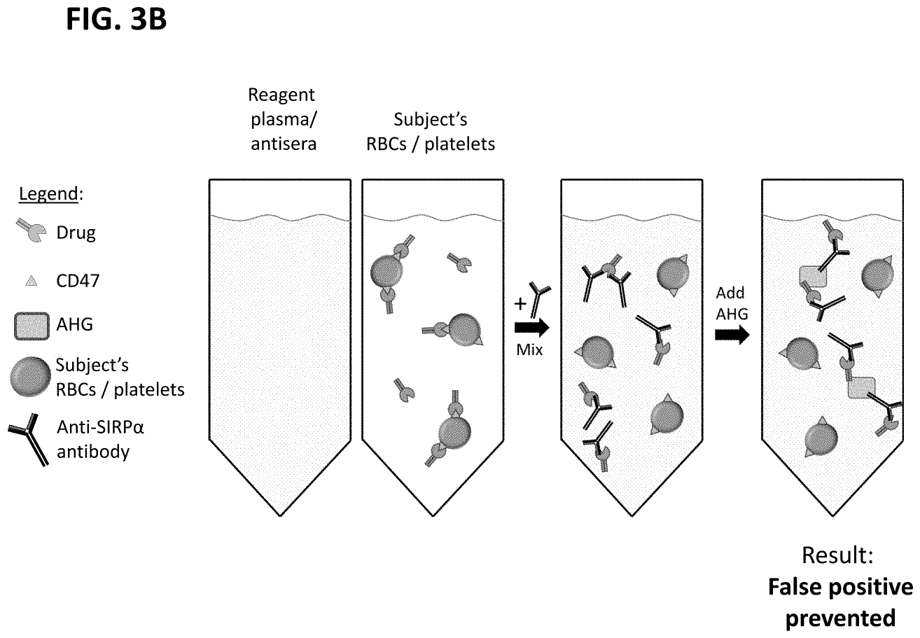

In some embodiments, the drug comprises (i) an antibody Fc region and (ii) a SIRPα variant that binds to human CD47, and the drug neutralizing agent is an anti-SIRPα antibody (or an antigen binding fragment thereof). In some embodiments, the method comprises (a) adding the anti-SIRPα antibody (or antigen binding fragment thereof) to a plasma sample from a subject who has received treatment with the drug, and (b) performing the serological assay of the plasma sample after step (a) using reagent RBCs and/or reagent platelets. As generically depicted in FIG. 3A, the anti-SIRPα antibody (or antigen binding fragment thereof) binds free drug present in the subject's plasma and reduces (or in some embodiments, eliminates) the amount of free drug available to CD47 on the surface of reagent RBCs and/or reagent platelets. The interference that would result from the binding of drug to the reagent RBCs and/or reagent platelets (as illustrated in FIG. 1B) is minimized (or, in some embodiments, eliminated), thus preventing a false positive result in the serological assay. In some embodiments, the anti-SIRPα antibody (or antigen binding fragment thereof) is added to the reagent RBCs and/or reagent platelets as well as to the subject's plasma before the serological assay is performed. Because the extracellular domains of SIRPα, SIRPβ, and SIRPγ are highly homologous, an anti-SIRPβ antibody that cross-reacts with SIRPα and/or an anti-SIRPγ antibody may cross-react with SIRPα. Thus, in some embodiments, an anti-SIRPβ antibody and/or an anti-SIRPγ antibody that cross-reacts with SIRPα is used in the method.

In some embodiments, the method comprises (a) adding the anti-SIRPα antibody (or antigen binding fragment thereof) to a blood sample from a subject who has received treatment with the drug, and (b) performing the serological assay of the blood sample after step (a) using reagent plasma/antisera. As generically depicted in FIG. 3B, the anti-SIRPα antibody (or fragment thereof) displaces the drug bound to CD47 on the surface of the subject's RBCs and/or platelets, thus reducing (and, in some embodiments, eliminating) interference caused by drug, e.g., as illustrated in FIG. 1D. In some embodiments, the anti-SIRPα antibody (or fragment thereof) is added to the reagent plasma/antisera as well as to the subject's RBCs and/or platelets before the serological assay is performed. Because the extracellular domains of SIRPα, SIRPβ, and SIRPγ are highly homologous, an anti-SIRPβ antibody and/or an anti-SIRPγ antibody may cross-react with SIRPα. Thus, in some embodiments, an anti-SIRPβ antibody that cross-reacts with SIRPα and/or an anti-SIRPγ antibody that cross-reacts with SIRPα is used in the method. In some embodiments, the anti-SIRPα antibody (or the anti-SIRPβ antibody that cross reacts with SIRPα or anti-SIRPγ antibody that cross-reacts with SIRPα) comprises an Fc domain (or a portion thereof) that does not bind to anti-human globulin reagent (AHG) used in the serological assay. Further details regarding serological assays, and reagents used in such assays, are provided elsewhere herein.

In some embodiments, the anti-SIRPα antibody (e.g., such as an anti-SIRPβ antibody that cross reacts with SIRPα and/or an anti-SIRPγ antibody that cross-reacts with SIRPα) is a full length antibody. In some embodiments, the antigen binding fragment of the anti-SIRPα antibody is, e.g., without limitation, a Fab, a Fab′, an F(ab′)₂, a Fab′-SH, an Fv, a diabody, a one-armed antibody, an scFv, an scFv-Fc, a single domain antibody, a single heavy chain antibody, etc. In some embodiments, the anti-SIRPα antibody (or antigen binding fragment thereof) is an ADA (anti-drug antibody) or a NAb (neutralizing antibody) that binds to the drug (i.e., the portion of the drug that comprises the SIRPα variant). In some embodiments, the anti-SIRPα antibody (or antigen binding fragment thereof) comprises a heavy chain variable domain (VH) that comprises the amino acid sequence of SEQ ID NO: 6 and a light chain variable domain (VL) that comprises the amino acid sequence of SEQ ID NO: 7. In some embodiments, the anti-SIRPα antibody (or antigen binding fragment thereof) comprises a heavy chain variable domain (VH) that comprises the amino acid sequence of SEQ ID NO: 21 and a light chain variable domain (VL) that comprises the amino acid sequence of SEQ ID NO: 22. In some embodiments, the anti-SIRPα antibody (or antigen binding fragment thereof) comprises a heavy chain variable domain (VH) that comprises the amino acid sequence of SEQ ID NO: 23 and a light chain variable domain (VL) that comprises the amino acid sequence of SEQ ID NO: 24.

(SEQ ID NO: 6)
DVQLVESGGG VVRPGESLRL SCAASGFSFS SYAMNWVRQA
PGEGLEWVSR INSGGGGTDY AESVKGRFTI SRDNSENTLY
LQMNSLRAED TAVYYCAKQY DWNSFFDYWG LGALVTVSS
(SEQ ID NO: 7)
ETVLTQSPAT LSVSPGERAT LSCRASQTVG SKLAWHQQKP
GQAPRLLIYD ATNRATGISD RFSGSGSGTD FTLTISSLQT
EDSAVYYCQQ YYYWPPYRFG GGTKVEIK
(SEQ ID NO: 21)
DVQLVESGGG VVRPGESLRL SCEASGFTFS SNAMSWVRQA
PGKGLEWVAG ISSGSDTYYG DSVKGRLTIS RDNSKNILYL
QMNSLTAEDT AVYYCARETW NHLFDYWGQG TLVTVSS
(SEQ ID NO: 22)
SYELTQPPSV SVSPGQTARI TCSGGSYSSY YYAWYQQKPG
QAPVTLIYSD DKRPSNIPER FSGSSSGTTV TLTISGVQAE
DEADYYCGGY DQSSYTNPFG GGTKLTVL
(SEQ ID NO: 23)
DVQLVESGGG VVRPGESLRL SCAASGFTFS SYDMNWVRQA
PGEGLEWVSL ISGSGEIIYY ADSVKGRFTI SRDNSKNTLY
LQMNSLRAED TAVYYCAKEN NRYRFFDDWG QGTLVTVSS
(SEQ ID NO: 24)
ETVLTQSPGT LTLSPGERAT LTCRASQSVY TYLAWYQEKP
GQAPRLLIYG ASSRATGIPD RFSGSGSGTE FTLTISSLQS
EDFAVYYCQQ YYDRPPLTFG GGTKVEIK

Other exemplary anti-SIRP antibodies (e.g., anti-SIRP antibodies, anti-SIRPβ antibodies, and/or anti-SIRPγ antibodies that cross-react with SIRPα) that can be used as drug neutralizing agents in the methods described herein are known in the art. Further details regarding such antibodies are provided in, e.g., WO 2018/057669; US-2018-0105600-A1; US20180312587; WO2018107058; WO2019023347; US20180037652; WO2018210795; WO2017178653; WO2018149938; WO2017068164; and WO2016063233, the contents of which are incorporated herein by reference in their entireties.

In some embodiments, the drug comprises (i) an antibody Fc region and (ii) a SIRPβ variant that binds to human CD47, and the drug neutralizing agent is an anti-SIRPβ antibody (or an antigen binding fragment thereof). In some embodiments, the method comprises (a) adding the anti-SIRPβ antibody (or an antigen binding fragment thereof) to a plasma sample from a subject who has received treatment with the drug, and (b) performing the serological assay of the plasma sample after step (a) using reagent RBCs and/or reagent platelets. In some embodiments, the anti-SIRPβ antibody (or an antigen binding fragment thereof) is added to the reagent RBCs and/or reagent platelets as well as to the subject's plasma before the serological assay is performed. In some embodiments, the anti-SIRPβ antibody is a full length antibody. In some embodiments, the antigen binding fragment of the anti-SIRPβ antibody is, e.g., without limitation, a Fab, a Fab′, an F(ab′)₂, a Fab′-SH, an Fv, a diabody, a one-armed antibody, an scFv, an scFv-Fc, a single domain antibody, a single heavy chain antibody, etc. Because the extracellular domains of SIRPα, SIRPβ, and SIRPγ are highly homologous, an anti-SIRPα antibody and/or an anti-SIRPγ antibody may cross-react with SIRPβ. Thus, in some embodiments, an anti-SIRPα antibody that cross reacts with SIRPβ and/or an anti-SIRPγ antibody that cross-reacts with SIRPβ is used in the method.

In some embodiments, the drug comprises (i) an antibody Fc region and (ii) a SIRPγ variant that binds to human CD47, and the drug neutralizing agent is an anti-SIRPγ antibody (or an antigen binding fragment thereof). In some embodiments, the method comprises (a) adding the anti-SIRPγ antibody (or an antigen binding fragment thereof) to a plasma sample from a subject who has received treatment with the drug, and (b) performing the serological assay of the plasma sample after step (a) using reagent RBCs and/or reagent platelets. In some embodiments, the anti-SIRPγ antibody (or an antigen binding fragment thereof) is added to the reagent RBCs and/or reagent platelets as well as to the subject's plasma before the serological assay is performed. In some embodiments, the anti-SIRPγ antibody is a full length antibody. In some embodiments, the antigen binding fragment of the anti-SIRPγ antibody is, e.g., without limitation, a Fab, a Fab′, an F(ab′)₂, a Fab′-SH, an Fv, a diabody, a one-armed antibody, an scFv, an scFv-Fc, a single domain antibody, a single heavy chain antibody, etc. Because the extracellular domains of SIRPα, SIRPβ, and SIRPγ are highly homologous, an anti-SIRPβ antibody and/or an anti-SIRPα antibody may cross-react with SIRPγ. Thus, in some embodiments, an anti-SIRPα antibody that cross reacts with SIRPγ and/or an anti-SIRPβ antibody that cross-reacts with SIRPγ is used in the method.

In some embodiments, the drug comprises an anti-CD47 antibody, and the drug neutralizing agent is an anti-idiotypic antibody (or an antigen binding fragment thereof) that binds the antigen-binding portion of the anti-CD47 antibody. In some embodiments, the method comprises (a) adding the anti-idiotypic antibody (or an antigen binding fragment thereof) to a plasma sample from a subject who has received treatment with the anti-CD47 antibody, and (b) performing the serological assay of the plasma sample after step (a) using reagent RBCs and/or reagent platelets. In some embodiments, the anti-idiotypic antibody (or an antigen binding fragment thereof) is added to the reagent RBCs and/or reagent platelets as well as to the subject's plasma before the serological assay is performed. In some embodiments, the anti-idiotypic antibody is a full length antibody. In some embodiments, the antigen binding fragment of the anti-idiotypic antibody is, e.g., without limitation, a Fab, a Fab′, an F(ab′)₂, a Fab′-SH, an Fv, a diabody, a one-armed antibody, an scFv, an scFv-Fc, a single domain antibody, a single heavy chain antibody, etc.

In some embodiments, the affinity of the drug for the drug neutralizing agent is higher than the affinity of the drug for human CD47 (e.g., human CD47 expressed on the surface regent RBCs) for drug. In some embodiments, the affinity of the drug neutralizing agent for the drug is at least about any one of 10-fold, 25-fold, 50-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold greater than the affinity of human CD47 for the drug.

In some embodiments, the amount of drug neutralizing agent added to the subject's plasma, to the sample comprising the subject's RBCs and/or platelets, to the reagent plasma/antisera, to the reagent RBCs, and/or to the reagent platelets is an amount sufficient to achieve an excess amount of drug neutralizing agent relative to the amount of drug in the subject's plasma or in the sample comprising the subject's RBCs and/or platelets. In some embodiments, the amount of drug neutralizing agent added to the subject's plasma, to the sample comprising the subject's RBCs and/and or platelets, to the reagent plasma/antisera, to the reagent RBCs, and/or to the reagent platelets is an amount sufficient to bind substantially all (such as all) of the drug in the subject's plasma or in the sample comprising the subject's RBCs and/or platelets. In some embodiments, the amount of drug neutralizing agent added to the subject's plasma, to the sample comprising the subject's RBCs and/or platelets, to the reagent plasma/antisera, to the reagent RBCs, and/or to the reagent platelets is an amount sufficient to achieve any one of about, e.g., a 2-fold, 5-fold, 10-fold, 25-fold, 50-fold, 75-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, 1000-fold, 1500-fold, 2000-fold, 2500-fold, 3000-fold, 3500-fold, 4000-fold, 4500-fold or 5000-fold molar excess (such as molar ratio or equivalent) of the drug neutralizing agent relative to the amount of drug in the subject's plasma or in the sample comprising the subject's RBC and/or platelets, including any range in between these values. In some embodiments, the amount of the drug neutralizing agent added to the subject's plasma, to the sample comprising the subject's RBC and/or platelets, to the reagent plasma/antisera, to the reagent RBCs, and/or to the reagent platelets is sufficient to achieve a concentration of any one of about 100 μg/ml, 200 μg/ml, 300 μg/ml, 400 g/ml, 500 μg/ml, 600 μg/ml, 700 μg/ml, 800 μg/ml, 900 μg/ml, 1 mg/ml, 1.25 mg/ml, 1.5 mg/ml, 1.75 mg/ml, 2 mg/ml, 2.25 mg/ml, 2.5 mg/ml, 2.75 mg/ml, 3 mg/ml, 3.25 mg/ml, 3.5 mg/ml, 3.75 mg/ml, 4 mg/ml 4.25 mg/ml. 4.5 mg/ml, 4.75 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 150 mg/ml, 200 mg/ml, 250 mg/ml, 300 mg/ml, 350 mg/ml, 400 mg/ml, 450 mg/ml, 500 mg/ml, 550 mg/ml, 600 mg/ml, 650 mg/ml, 700 mg/ml, 750 mg/ml, 800 mg/ml, 850 mg/ml, 900 mg/ml, 1000 mg/ml, 1100 mg/ml, 1150 mg/ml, 1200 mg/ml, 1250 mg/ml, 1300 mg/ml, 1350 mg/ml, 1400 mg/ml, 1450 mg/ml, 1500 mg/ml, 1550 mg/ml, 1600 mg/ml, 1650 mg/ml, 1700 mg/ml, 1750 mg/ml, 1800 mg/ml, 1850 mg/ml, 1900 mg/ml, or 2000 mg/ml of the drug neutralizing agent, including any range in between these values. In some embodiments, at least about any one of 5 g, 10 g, 50 g, 100 μg, 200 μg, 300 μg, 400 μg, 500 μg, 600 μg, 700 μg, 800 μg, 900 μg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg. 4.5 mg, 4.75 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1000 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, or 2000 mg of the drug neutralizing agent is added to the subject's plasma, to the sample comprising the subject's RBCs and/or platelets, to the reagent plasma/antisera, to the reagent RBCs, and/or to the reagent platelets. In some embodiments, the drug neutralizing agent is added to the subject's plasma, to the sample comprising the subject's RBCs and/or platelets, to the reagent plasma/antisera, and/or to the reagent RBCs in an amount to achieve at least about any one of 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold excess of the K_{D} of the drug for human CD47, including any range in between these values. In some embodiments, the drug neutralizing agent is added to the subject's plasma, to the sample comprising the subject's RBCs and/or platelets, to the reagent plasma/antisera, and/or to the reagent RBCs in an amount to achieve at least about any one of 500-fold, 1000-fold, 5000-fold, 10⁴-fold, 10⁵-fold, 10⁶-fold, 10⁷-fold, 10⁸-fold, 10⁹-fold, or 10¹⁰-fold excess of the K_{D} of the drug for human CD47, including any range in between these values. In some embodiments the drug neutralizing agent is added to the subject's plasma, to the sample comprising the subject's RBCs and/or platelets, to the reagent plasma/antisera, and/or to the reagent RBCs in an amount to achieve about any one of 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold excess of the K_{D} of the drug neutralizing agent for the drug, including any range in between these values. In some embodiments the drug neutralizing agent is added to the subject's plasma, to the sample comprising the subject's RBCs and/or platelets, to the reagent plasma/antisera, and/or to the reagent RBCs in an amount to achieve about any one of 500-fold, 1000-fold, 5000-fold, 10⁴-fold, 10⁵-fold, 10⁶-fold, 10⁷-fold, 10⁸-fold, 10⁹-fold, or 10¹⁰-fold excess of the K_{D} of the drug neutralizing agent for the drug, including any range in between these values.

In some embodiments, the drug neutralizing agent is any agent that is capable of binding the Fc region of the drug. In some embodiments, the drug neutralizing agent is SP-A (Surfactant Protein A) or SP-D (Surfactant Protein D).

In some embodiments, the method comprises using two or more drug neutralizing agents described herein. For example, in some embodiments, a method provided herein comprises adding, e.g., two or more different CD47 variants (or fragments thereof capable of binding to the drug), two or more different anti-SIRPα antibodies (or antigen binding fragments thereof), two or more different anti-SIRPβ antibodies (or antigen binding fragments thereof), two or more different anti-SIRPγ antibodies (or antigen binding fragments thereof), two or more different anti-idiotypic anti-CD47 antibodies (or antigen binding fragments thereof), or two or more agents that bind the Fc region of the drug to the plasma sample from a subject who has received treatment with the drug. In some embodiments, a method provided herein comprises adding a combination of different drug neutralizing agents, e.g., a combination comprising a soluble CD47 agent and/or an anti-SIRPα antibody (or antigen an binding fragment thereof), and/or an anti-SIRP antibody (or antigen an binding fragment thereof), and/or an anti-SIRPβ2 antibody (or antigen an binding fragment thereof), and/or an anti-SIRPγ antibody (or antigen an binding fragment thereof) and/or an anti-idiotypic anti-CD47 antibody (or antigen an binding fragment thereof) and/or an agent that bind the Fc region of the drug to the plasma sample from a subject who has received treatment with the drug. In some embodiments, the method comprises adding two or more different anti-SIRPγ antibodies (or antigen binding fragments thereof) to a sample of the subject's plasma, to a sample comprising the subject's RBC and/or platelets, to the reagent plasma/antisera, to the reagent RBCs, and/or to the reagent platelets.

### B. Methods of Using Cell Binding Agents to Mitigate Interference in a Pre-Transfusion Serological Assay

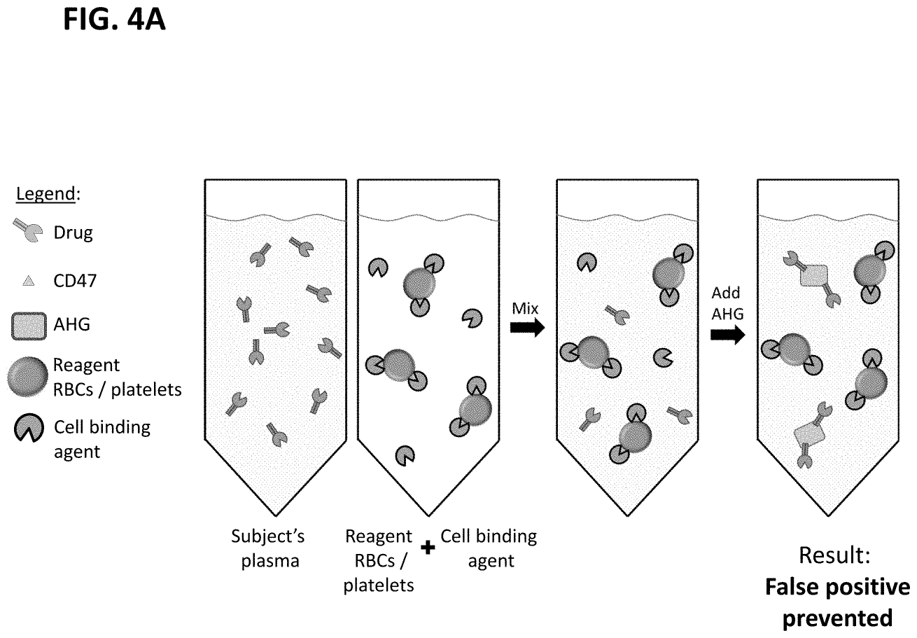

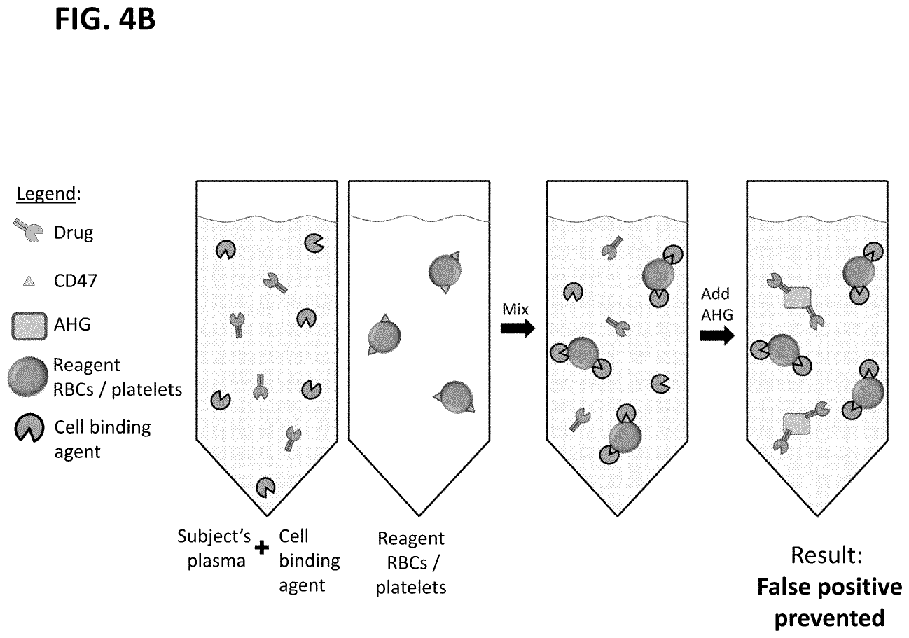

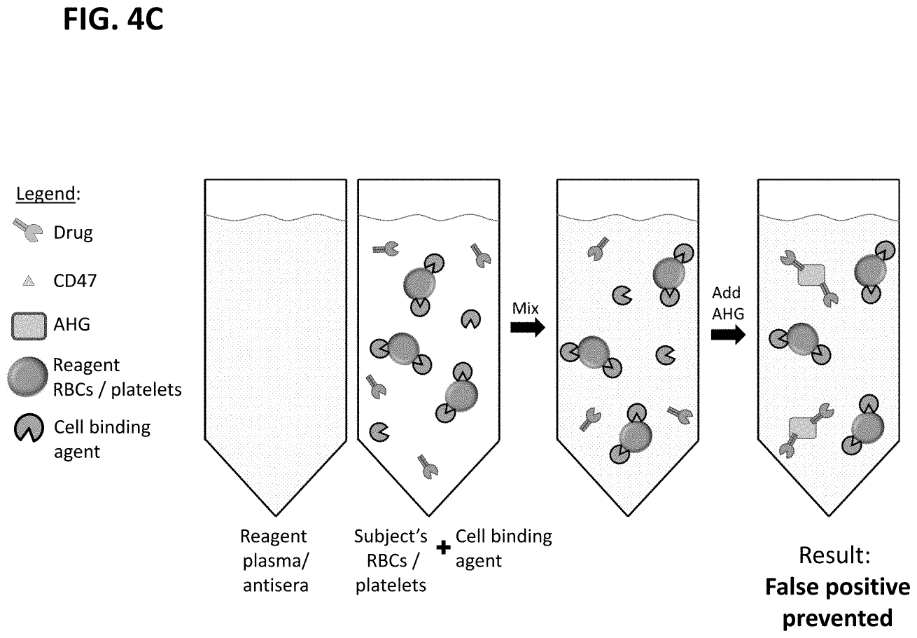

In some embodiments, the method comprises (a) adding a cell binding agent that binds human CD47 and does not comprise an antibody Fc region to reagent RBCs (i.e., RBCs that are known to express a particular cell surface antigen, or group of cell surface antigens) and/or reagent platelets (i.e., platelets that are known to express a particular cell surface antigen, or group of cell surface antigens) and (b) performing the serological assay of a plasma sample using the reagent RBCs and/or reagent platelets after step (a), wherein the plasma sample is from a subject who has received treatment with a drug, and wherein the drug comprises (i) an antibody Fc region and (ii) a moiety that binds to human CD47. Such embodiments are generically depicted in FIG. 4A. As shown in FIG. 4A, the cell binding agent binds the CD47 expressed on the surface of the reagent RBCs (and/or reagent platelets), and blocks the drug from binding the reagent RBCs (and/or reagent platelets), thereby minimizing (or, in some embodiments, eliminating) the interference that would result from the binding of drug to the reagent RBCs and/or reagent platelets (as illustrated in FIG. 1B). In some embodiments, the cell binding agent is added to the subject's plasma as well as to the reagent RBCs and/or reagent platelets before the serological assay is performed.

In some embodiments, the method comprises (a) adding a cell binding agent that binds to human CD47 and does not comprise an antibody Fc region to a plasma sample from a subject who has received treatment with a drug and (b) performing the serological assay of the plasma sample after step (a) using the reagent RBCs and/or reagent platelets, wherein the drug comprises (i) an antibody Fc region and (ii) a moiety that binds to human CD47. As shown in FIG. 4B, the cell binding agent competes with the drug for binding to the CD47 expressed on the surface of the reagent RBCs (and/or reagent platelets), thereby minimizing (or, in some embodiments, eliminating) the interference that would result from the binding of drug to the reagent RBCs and/or reagent platelets (as illustrated in FIG. 1B).

In some embodiments, the method comprises (a) adding a cell binding agent that binds to human CD47 and does not comprise an antibody Fc region to a blood sample from a subject who has received treatment with a drug and (b) performing the serological assay of the blood sample after step (a) using the reagent plasma/antisera, wherein the drug comprises (i) an antibody Fc region and (ii) a moiety that binds to human CD47. As shown in FIG. 4C, the cell binding agent competes with the drug for binding to the CD47 expressed on the surface of the subject's RBCs and/or platelets, thereby minimizing (or, in some embodiments, eliminating) the interference that would result from the binding of drug to the subject's RBCs and/or platelets (as illustrated in FIG. 1D). In some embodiments, the cell binding agent is added to the reagent plasma/antisera as well as to the blood sample from the subject before the serological assay is performed.

In some embodiments, the cell binding agent is any agent that binds CD47 and blocks the drug from binding CD47. As discussed above, the cell binding agent does not comprise an antibody Fc region. In alternative embodiments, the cell binding agent comprises an antibody Fc region variant that does not bind AHG.

In some embodiments, the method is performed in solution, e.g., wherein the cell binding agent is soluble. In some embodiments, the cell binding agent is immobilized to a solid phase before the method is performed via adsorption to a matrix or surface, covalent coupling, or non-covalent coupling. In some embodiments, the cell binding agent is capable of binding CD47 following immobilization to the solid phase. The solid phase or surface used for immobilization can be any inert support or carrier that is essentially water insoluble and useful in immunoassays, including supports in the form of, for example, surfaces, particles, porous matrices, cellulose polymer sponge (ImmunoCAP®, Phadia), and the like. Examples of commonly used supports include small sheets, Sephadex, polyvinyl chloride, plastic beads, microparticles, assay plates, or test tubes manufactured from polyethylene, polypropylene, polystyrene, and the like. In some embodiments, the cell binding agent is coated on a microtiter plate, such as a multi-well microtiter plate that can be used to analyze multiple samples simultaneously.

In some embodiments, the cell binding agent comprises a SIRPα or a SIRPγ. In some embodiments, the cell binding agent comprises a fragment of a SIRPα or a SIRPγ that is capable of binding to CD47 (e.g., the extracellular domain of a wild type SIRPα (“WT SIRPα-ECD”) or the D1 domain thereof, e.g., the extracellular domain of a wild type SIRPγ (“WT SIRPγ-ECD”) or the D1 domain thereof). In some embodiments, the cell binding agent comprises a human SIRPα, a human SIRPγ, a mouse SIRPα, a mouse SIRPγ, a rat SIRPα, a rat SIRPγ, a rhesus SIRPα, a rhesus SIRPγ, a cynomolgus SIRPα, a cynomolgus SIRPγ, a SIRPα of any origin, or a SIRPγ of any origin, provided that the SIRPα or SIRPγ is capable of binding to CD47 (e.g., human CD47 expressed on the surface of reagent RBCs and/or reagent platelets). In some embodiments, the cell binding agent comprises a fragment of a human SIRPα, human SIRPγ, mouse SIRPα, mouse SIRPγ, rat SIRPα, rat SIRPγ, rhesus SIRPα, rhesus SIRPγ, cynomolgus SIRPα, cynomolgus SIRPγ, SIRPα of any origin, or SIRPγ of any origin, provided that the fragment is capable of binding to CD47 (e.g., human CD47 expressed on the surface of reagent RBCs and/or reagent platelets). In some embodiments, the cell binding agent is a SIRPα variant (or a fragment thereof, such as a variant of a WT SIRPα-ECD or the D1 domain thereof), a soluble SIRPγ variant (or a fragment thereof, such as a variant of a WT SIRPβ-ECD or the D1 domain thereof), or a soluble SIRPγ variant (or a fragment thereof, such as a variant of a WT SIRPγ-ECD or the D1 domain thereof) that is capable of binding to human CD47. In some embodiments, the SIRPα variant, SIRPγ variant, or SIRPγ variant (or fragment of any one of the preceding that is capable of binding CD47) comprises one or more amino acid substitution(s), deletion(s), insertion(s), N-terminal addition(s) and/or C-terminal addition(s) relative to a wild type SIRPα, a wild type SIRPγ, or a wild SIRPγ, respectively. In some embodiments, the one or more amino acid substitution(s), deletion(s), insertion(s), N-terminal addition(s) and/or C-terminal addition(s) present in the SIRPα variant, SIRPγ variant, or SIRPγ variant (or fragment of any one of the preceding that is capable of binding CD47) alter the glycosylation pattern of the SIRPα variant, soluble SIRPγ variant, or soluble SIRPγ variant relative to a wild type SIRPα, a wild type SIRPγ, or a wild type SIRPγ, respectively. In some embodiments, the one or more amino acid substitution(s), deletion(s), insertion(s), N-terminal addition(s) and/or C-terminal addition(s) present in the SIRPα variant, SIRPγ variant, or SIRPγ variant (or fragment of any one of the preceding that is capable of binding CD47) increase the affinity of the SIRPα variant, SIRPγ variant, or SIRPγ variant for human CD47, relative to a wild type SIRPα, SIRPγ, or SIRPγ, respectively. In some embodiments, the cell binding agent is a monomer (e.g., a wild type SIRPα monomer, a wild type SIRPγ monomer, a SIRPα variant monomer, a SIRPγ variant monomer, a SIRPγ monomer, or a fragment of any one of the preceding that is capable of binding CD47). In some embodiments, the cell binding agent is a dimer (e.g., a homodimer or a heterodimer comprising any combination of, e.g., a wild type SIRPα, a wild type SIRPγ, a SIRPα variant, a SIRPγ variant, a SIRPγ variant, or a fragment of any one of the preceding that is capable of binding CD47). In some embodiments, the cell binding agent is an oligomer (e.g., a homoöligomer or a heteroöligomer comprising any combination of, e.g., one or more wild type SIRPαs, wild type SIRPγs, SIRPα variants, a SIRPγ variants, a SIRPγ variants, and/or fragments of any one of the preceding that are capable of binding CD47.

In some embodiments, the cell binding agent comprises a SIRPα variant, a SIRPβ variant, or a SIRPγ variant. In some embodiments, the SIRPα variant, SIRPβ variant, or SIRPγ variant comprises the amino acid sequence of any one of SEQ ID NOs: 8-16 below.

(SEQ ID NO: 8)
EEELQIIQPD KSVLVAAGET ATLRCTITSL FPVGPIQWFR
GAGPGRVLIY NQRQGPFPRV TTVSDTTKRN NMDFSIRIGA
ITPADAGTYY CIKFRKGSPD DVEFKSGAGT ELSVRAKPS
(SEQ ID NO: 9)
EEELQIIQPD KSVLVAAGET ATLRCTITSL FPVGPIQWFR
GAGPGRELIY NQREGPFPRV TTVSDTTKRN NMDFSIRIGA
ITPADAGTYY CVKFRKGSPD DVEFKSGAGT ELSVRAKPS
(SEQ ID NO: 10)
EEELQIIQPD KSVLVAAGET ATLRCTITSL FPVGPIQWFR
GAGPGRVLIY NQREGPFPRV TTVSDTTKRN NMDFSIRIGA
ITPADAGTYY CIKFRKGSPD DVEFKSGAGT ELSVRAKPS
(SEQ ID NO: 11)
EDELQIIQPE KSVSVAAGES ATLRCAITSL FPVGPIQWFR
GAGAGRVLIY NQRQGPFPRV TTVSETTKRN NLDFSISISN
ITPADAGTYY CIKFRKGSPD DVEFKSGAGT ELSVRAKPS
(SEQ ID NO: 12)
EEELQIIQPD KSISVAAGES ATLHCTITSL FPVGPIQWFR
GAGPGRVLIY NQRQGPFPRV TTVSDTTKRN NMDFSIRISN
ITPADAGTYY CIKFRKGSPD DVEFKSGAGT ELSVRAKPS
(SEQ ID NO: 13)
EEELQIIQPE KLLLVTVGKT ATLHCTITSL FPVGPIQWFR
GVGPGRVLIY NQRDGPFPRV TTVSDGTKRN NMDFSIRISS
ITPADVGTYY CVKFRKGTPE DVEFKSGPGT EMALGAKPS
(SEQ ID NO: 14)
EEELQIIQPE KLLLVTVGKT ATLHCTITSL FPVGPIQWFR
GVGPGRVLIY NQKDGPFPRV TTVSDGTKRN NMDFSIRISS
ITPADVGTYY CVKFRKGSPE DVEFKSGPGT EMALGAKPS
(SEQ ID NO: 15)
EEELQIIQPE KLLLVTVGKT ATLHCTITSL FPVGPIQWFR
GVGPGRVLIY NQKDGHFPRV TTVSDGTKRN NMDFSIRISS
ITPADVGTYY CVKFRKGSPE DVEFKSGPGT EMALGAKPS
(SEQ ID NO: 16)
EEELQIIQPE KLLLVTVGKT ATLHCTITSH FPVGPIQWFR
GVGPGRVLIY NQKDGHFPRV TTVSDGTKRN NMDFSIRISS
ITPADVGTYY CVKFRKGSPE DVEFKSGPGT EMALGAKPS
(SEQ ID NO: 17)
EEELQIIQPE KLLLVTVGKT ATLHCTITSL FPVGPVLWFR
GVGPGRVLIY NQRQGPFPRV TTVSDTTKRN NMDFSIRISS
ITPADVGTYY CVKFRKGTPE DVEFKSGPGT EMALGAKPS
(SEQ ID NO: 18)
EEELQIIQPE KLLLVTVGKT ATLHCTITSL FPVGPIQWFR
GVGPGRELIY NAREGRFPRV TTVSDLTKRN NMDFSIRISS
ITPADVGTYY CVKFRKGSPE DVEFKSGPGT EMALGAKPS
(SEQ ID NO: 19)
EEELQIIQPE KLLLVTVGKT ATLHCTITSL LPVGPIQWFR
GVGPGRELIY NQRDGPFPRV TTVSDGTKRN NMDFSIRISS
ITPADVGTYY CVKFRKGTPE DVEFKSGPGT EMALGAKPS
(SEQ ID NO: 20)
EEELQIIQPD KSVLVAAGET ATLRCTITSL FPVGPIQWFR
GAGPGRVLIY NQRQGPFPRV TTVSDTTKRN NMDFSIRIGN
ITPADAGTYY CIKFRKGSPD DVEFKSGAGT ELSVRAKPS

Other exemplary soluble SIRPα variants, soluble SIRPβ variants, and SIRPγ variants are known in the art and are described in WO 2013/109752; US 2015/0071905; U.S. Pat. No. 9,944,911; WO 2016/023040; WO 2017/027422; US 2017/0107270; U.S. Pat. Nos. 10,259,859; 9,845,345; WO2016187226; US20180155405; WO2017177333; WO2014094122; US2015329616; US20180312563; WO2018176132; WO2018081898; WO2018081897; US20180141986A1; and EP3287470A1, the contents of which are incorporated herein by reference in their entireties.

In some embodiments, the cell binding agent is a fragment of an anti-CD47 antibody that does not comprise an Fc region. In some embodiments, such fragments include, without limitation, e.g., a Fab, a Fab′, a Fab′-SH, an F(ab′)2, an Fv, an scFv, or a diabody. Exemplary anti-CD47 antibody fragments that can be used with the methods provided herein include, but are not limited to, e.g., murine 5F9 (see Liu et al. (2015) PLoS One. 10(9): e0137345), Hu5F9-G4 (Forty Seven, Inc.), B6H12.2, CC2C6, 8H7, BRIC 126, and others. In some embodiments, the anti-CD47 antibody comprises an Fc region that does not bind AHG.

In some embodiments, the affinity of the cell binding agent for human CD47 is higher than the affinity of the drug for human CD47 (e.g., human CD47 expressed on the surface regent RBCs and/or platelets) for drug. In some embodiments, the affinity of the cell binding agent for human CD47 is at least about any one of 10-fold, 25-fold, 50-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold greater than the affinity of the drug for CD47, including any range in between these values.

In some embodiments, the amount of cell binding agent added to the subject's plasma, to the sample comprising the subject's RBCs/platelets, to the reagent plasma/antisera, to the reagent RBCs and/or to the reagent platelets is an amount sufficient to achieve an excess amount of cell binding agent relative to the amount of CD47 expressed on the subject's RBCs/platelets, the reagent RBCs and/or reagent platelets. In some embodiments, the amount of cell binding agent added to the subject's plasma, to the sample comprising the subject's RBCs/platelets, to the reagent plasma/antisera, to the reagent RBCs and/or to the reagent platelets is an amount sufficient to bind substantially all (such as all) of the CD47 expressed on the subject's RBCs/platelets, on the reagent RBCs and/or reagent platelets. In some embodiments, the amount of cell binding agent added to the subject's plasma, to the sample comprising the subject's RBC/platelets, to the reagent plasma/antisera, to the reagent RBCs and/or to the reagent platelets is an amount sufficient to achieve any one of about, e.g., a 2-fold, 5-fold, 10-fold, 25-fold, 50-fold, 75-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, 1000-fold, 1500-fold, 2000-fold, 2500-fold, 3000-fold, 3500-fold, 4000-fold, 4500-fold or 5000-fold molar excess (such as molar ratio or equivalent) of the cell binding agent relative to the amount of CD47 expressed on the subject's RBCs/platelets, on the reagent RBCs and/or reagent platelets, including any range in between these values. In some embodiments, the amount of cell binding agent added to the subject's plasma, to the sample comprising the subject's RBCs/platelets, to the reagent plasma/antisera, to the reagent RBCs and/or to the reagent platelets is sufficient to achieve a concentration of any one of about 100 g/ml, 200 μg/ml, 300 μg/ml, 400 μg/ml, 500 μg/ml, 600 μg/ml, 700 μg/ml, 800 μg/ml, 900 μg/ml, 1 mg/ml, 1.25 mg/ml, 1.5 mg/ml, 1.75 mg/ml, 2 mg/ml, 2.25 mg/ml, 2.5 mg/ml, 2.75 mg/ml, 3 mg/ml, 3.25 mg/ml, 3.5 mg/ml, 3.75 mg/ml, 4 mg/ml 4.25 mg/ml. 4.5 mg/ml, 4.75 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 150 mg/ml, 200 mg/ml, 250 mg/ml, 300 mg/ml, 350 mg/ml, 400 mg/ml, 450 mg/ml, 500 mg/ml, 550 mg/ml, 600 mg/ml, 650 mg/ml, 700 mg/ml, 750 mg/ml, 800 mg/ml, 850 mg/ml, 900 mg/ml, 1000 mg/ml, 1100 mg/ml, 1150 mg/ml, 1200 mg/ml, 1250 mg/ml, 1300 mg/ml, 1350 mg/ml, 1400 mg/ml, 1450 mg/ml, 1500 mg/ml, 1550 mg/ml, 1600 mg/ml, 1650 mg/ml, 1700 mg/ml, 1750 mg/ml, 1800 mg/ml, 1850 mg/ml, 1900 mg/ml, or 2000 mg/ml of the cell binding agent, including any range in between these values. In some embodiments, at least about any one of 5 g, 10 g, 50 g, 100 μg, 200 μg, 300 μg, 400 μg, 500 μg, 600 μg, 700 μg, 800 μg, 900 μg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg. 4.5 mg, 4.75 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1000 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, or 2000 mg of the cell binding agent is added to the subject's plasma, to the sample comprising the subject's RBC, to the reagent plasma/antisera, and/or to the reagent RBCs. In some embodiments the amount of cell binding agent added to the subject's plasma, to the sample comprising the subject's RBCs/platelets, to the reagent plasma/antisera, to the reagent RBCs and/or to the reagent platelets is at least about any one of about 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold excess of the K_{D} of the drug for human CD47, including any range in between these values. In some embodiments the amount of cell binding agent added to the subject's plasma, to the sample comprising the subject's RBCs/platelets, to the reagent plasma/antisera, to the reagent RBCs and/or to the reagent platelets is at least about any one of 500-fold, 1000-fold, 5000-fold, 10⁴-fold, 10⁵-fold, 10⁶-fold, 10⁷-fold, 10⁸-fold, 10⁹-fold, or 10¹⁰-fold excess of the K_{D} of the drug for human CD47, including any range in between these values. In some embodiments the amount of cell binding agent added to the subject's plasma, to the sample comprising the subject's RBCs/platelets, to the reagent plasma/antisera, to the reagent RBCs and/or to the reagent platelets is at least about any one of 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold excess of the K_{D} of the cell binding agent for human CD47, including any range in between these values. In some embodiments the amount of cell binding agent added to the subject's plasma, to the sample comprising the subject's RBCs/platelets, to the reagent plasma/antisera, to the reagent RBCs and/or to the reagent platelets is at least about any one of 500-fold, 1000-fold, 5000-fold, 10⁴-fold, 10⁵-fold, 10⁶-fold, 10⁷-fold, 10⁸-fold, 10⁹-fold, or 10¹⁰-fold excess of the K_{D} of the cell binding agent for human CD47, including any range in between these values.

### C. Exemplary Drugs

The methods provided herein reduce (or, in some embodiments, eliminate) interference in serological assays caused by a the presence of a drug comprising (i) an antibody Fc region and (ii) a moiety that binds to human CD47 in a sample comprising plasma or RBCs/platelets obtained from a subject who has received treatment with the drug. In some embodiments, the drug comprises an IgG Fc region, such as a human IgG Fc region, e.g., an IgG1, IgG2, or an IgG4 Fc region. In some embodiments, the drug comprises a modified Fc region (such as a modified IgG Fc region) that comprises one or more amino acid substitution(s), deletion(s), insertion(s), N-terminal addition(s) and/or C-terminal addition(s) relative to a wild type human Fc region (e.g., a wild type human IgG Fc region). Exemplary Fc regions are described in WO2017177333; WO2014094122; US2015329616, WO 2017/027422; US 2017/0107270; and U.S. Pat. No. 10,259,859, the contents of which are incorporated herein by reference in their entirety.

In some embodiments, the moiety that binds to human CD47 is a wild type SIRPα that lacks a transmembrane domain (e.g., the extracellular domain of any wild type SIRPα that is capable of binding human CD47). In some embodiments, the moiety that binds to human CD47 is a SIRPα variant that is capable of binding human CD47 and lacks a transmembrane domain. In some embodiments, the SIRPα variant comprises one or more amino acid substitution(s), deletion(s), insertion(s), N-terminal addition(s) and/or C-terminal addition(s) relative to extracellular domain of a wild-type SIRPα. In some embodiments, the SIRPα variant is a SIRPα-d1 domain variant. In some embodiments the affinity of the SIRPα variant for human CD47 is higher than the affinity of a wild type SIRPα for human CD47.

In some embodiments, the moiety that binds to human CD47 is a wild type SIRPγ that lacks a transmembrane domain (e.g., the extracellular domain of any wild type SIRPγ that is capable of binding human CD47). In some embodiments, the moiety that binds to human CD47 is a SIRPγ variant that is capable of binding human CD47 and lacks a transmembrane domain. In some embodiments, the SIRPγ variant comprises one or more amino acid substitution(s), deletion(s), insertion(s), N-terminal addition(s) and/or C-terminal addition(s) relative to extracellular domain of a wild-type SIRPγ. In some embodiments, the SIRPγ variant is a SIRPγ-d1 domain variant. In some embodiments the affinity of the SIRPγ variant for human CD47 is higher than the affinity of a wild type SIRPγ for human CD47.

In some embodiments, the moiety that binds to human CD47 is a SIRPγ variant that is capable of binding human CD47 and lacks a transmembrane domain. In some embodiments, the SIRPγP variant comprises one or more amino acid substitution(s), deletion(s), insertion(s), N-terminal addition(s) and/or C-terminal addition(s) relative to extracellular domain of a wild-type SIRPγ. In some embodiments, the SIRPγ variant is a SIRPα-d1 domain variant.

Exemplary SIRPα variants, SIRPγ variants, and SIRPγ variants are known in the art and are described in WO 2013/109752; US 2015/0071905; U.S. Pat. No. 9,944,911; WO 2016/023040; WO 2017/027422; US 2017/0107270; U.S. Pat. Nos. 10,259,859; 9,845,345; WO2016187226; US20180155405; WO2017177333; WO2014094122; US2015329616; US20180312563; WO2018176132; WO2018081898; WO2018081897; US20180141986A1; and EP3287470A1, the contents of which are incorporated herein by reference in their entireties.

In some embodiments of any of the methods described above, the drug is an anti-CD47 antibody. In some embodiments, the anti-CD47 antibody is AO-176, CC-90002, Hu5F9-G4 (also referred to as 5F9), SHR-1603, NI-1701, SRF231, TJC4, or IBI188. Details regarding these and other therapeutic anti-CD47 antibodies are provided in WO2018175790A1; US20180142019; US20180171014; US20180057592; US20170283498, U.S. Pat. Nos. 9,518,116; 9,518,117; US20150274826; US20160137733; U.S. Pat. No. 9,221,908; US20140161799; US20160137734; WO2015191861; WO2014093678; WO2014123580; WO2013119714; U.S. Pat. No. 9,045,541; WO2016109415; WO2018183182; WO2018009499; WO2017196793; U.S. Pat. No. 9,663,575; US20140140989; WO2018237168; US20180037652; US20190023784; WO2018095428; EP3411071; WO2019042285; WO2016081423; WO2011076781; WO2012172521; WO2014087248; US20140303354; WO2016156537; US20160289727; US20190062428; US20180201677; U.S. Pat. No. 9,352,037; US20170044258; U.S. Pat. No. 9,650,441; and US20180105591 Serological Assays for Pre-Transfusion Testing

Pre-transfusion testing is performed to ensure that the blood product intended for transfusion is compatible with the blood of the subject (i.e., the recipient of the transfusion). Pre-transfusion testing encompasses the serological assays that are used to confirm ABO compatibility between donor blood and recipient blood, as well as those that are used to detect most clinically significant RBC/platelet alloantibodies that react with antigens on donor RBCs and/or donor platelets (ref. Technical Manual, 18th ed, AABB, Bethesda, Md., 2014). Other exemplary blood group antigens for which serological assays are performed to determine donor/recipient transfusion compatibility include, without limitation, e.g., Kell blood group antigens, Duffy blood group antigens, Knops blood group antigens, Cartwright blood group antigens, Scianna blood group antigens, Indian blood group antigens, Rhesus blood group antigens, Dombrock blood group antigens, Landsteiner-Wiener blood group antigens, and VEL blood group antigens. The methods provided herein reduce or prevent drug interference (e.g., interference by a drug comprising (i) an antibody Fc region and (ii) a moiety that binds to human CD47) in a number of serological assays known in the art. Exemplary serological assays in which the methods can be used include (but are not limited to) those described in further detail below.

Typically, serological assays are performed using samples comprising, e.g., non-hemolyzed blood, plasma (e.g., a plasma sample that has been anticoagulated in EDTA), clotted blood, or serum from a subject who is in need of the transfusion (e.g., a subject who has received treatment with a drug comprising (i) an antibody Fc region and (ii) a moiety that binds to human CD47. In general, the subject's ABO group and Rh type are determined first. Next, an antibody screening method is used to detect any clinically significant unexpected non-ABO blood group antibodies that may be present in the subject's plasma. If the screening test reveals the presence of such an antibody, the specificity of that antibody is determined using an antibody identification panel. After the specificity of the antibody is identified, donor units of the appropriate ABO group and Rh type are screened for the corresponding antigen. Units that are negative for that antigen are crossmatched with the subject who is in need of the transfusion to ensure compatibility.

Serological assays can be performed in a tube, on a slide, on a gel column or in microtiter well plates, and hemolysis and agglutination are signals that indicate a positive (incompatible) test result. Agglutination, a reaction reflecting linkage of adjacent RBCs that are coated with antibody, can be scored macroscopically and/or microscopically and on scale from 0-4+ in the most commonly used tube methods. A score of zero indicates no reactivity and is characterized by smooth and easily dispersed cells. A score of 4+ indicates strong reactivity and is characterized by one solid agglutinate that is not easily dispersed. Scores of 1+, 2+, or 3+ indicate intermediate levels of reactivity, characterized by gradually increasing size of agglutinates with higher scores. Similar principles of agglutination scoring can be applied when the serological tests are conducted using gel columns with anti IgG antibody in the column (gel card) or microtiter well plates with bound red blood cell antigens (solid phase). Various techniques are currently available for the detection of antibody-RBC antigen interaction with varying sensitivities. In some embodiments, serological assays are performed manually. In some embodiments, serological assays are performed via automated machine.

For example, immediate-spin (IS) (also known as “immediate spin crossmatch”) is an assay that entails mixing, e.g., reagent plasma/antisera (i.e., plasma containing antibodies against a known RBC and/or platelet surface antigen) and the subject's blood cells, immediately centrifuging the mixture for about 15-30 seconds at room temperature or at 37° C., and visually examining the tube for direct agglutination. Direct agglutination indicates that there is a strong interaction between an antibody in the plasma and an RBC surface antigen. Alternatively, the subject's plasma and reagent RBC (i.e., RBC that are known to express a particular cell surface antigen, or group of cell surface antigens) and/or regent platelets (i.e., platelets that are known to express a particular cell surface antigen, or group of cell surface antigens) can be mixed, centrifuged, and assessed visually for direct agglutination.

Anti-human globulins (AHGs) are used to detect antibody-bound RBC that do not produce direct agglutination. AHG are secondary anti-human globulin antibodies that have been produced in another species. AHG reagents can be specific for a single class of human Ig (such as IgG), or polyspecific, i.e., capable of binding to multiple human Ig classes (e.g., IgG, IgM, IgA) and to complement. AHG sera may be used in a direct antiglobulin test (DAT) and/or in an indirect antiglobulin test (IAT). The DAT demonstrates in vivo sensitization of red cells and is performed by directly testing a sample of washed patient red cells with AHG. An IAT demonstrates in vitro reactions between red cells and antibodies. In an IAT, serum (or plasma) is incubated with red cells, which are then washed to remove unbound globulins. The presence of agglutination with the addition of AHG indicates antibody binding to a specific red cell antigen. Some methods involve addition of potentiator reagents (enhancement) such as saline, albumin, low ionic strength saline (LISS), or polyethylene glycol (PEG), and the samples are then incubated at 37° C. for 10-60 minutes prior to the AHG test.

ABO typing involves testing the recipient's red blood cells for the presence of A and B antigens using anti-A and anti-B antisera (forward grouping). Testing of the recipient plasma for the presence of anti-A and anti-B using known Type A and Type B red blood cells (reverse grouping) is also part of routine ABO blood group testing.

The Rh (D) type of the transfusion recipient is determined by testing recipient red blood cells with anti-D. ABO grouping is typically tested using immediate spin (IS).

Alloantibodies to antigens that are not present on an individual's red blood cells may develop in anyone who has been exposed to foreign red blood cell antigens through pregnancy or transfusion. To detect antibodies to non-group A or B antigens, a sample of the patient's plasma or serum is tested against selected commercial Type O red blood cells that express the majority of clinically significant antigens, other than A and B.

In cases of positive antibody screening, further serological testing is conducted with an expanded panel of commercial Type O reagent RBCs for the identification of clinically significant antibodies is required. Then, once the specificity of the antibody is known, donor units must be screened for the corresponding antigen to select those units that lack the antigen.

Antigen typing (phenotyping) of the recipient red blood cells may also be performed to determination of which red blood cell antibodies an individual is likely to develop. Serological assay for RBC phenotyping involves mixing recipient cells with commercial reagent anti-sera containing specific antibodies.

An IAT without and with enhancement (e.g. saline, LISS, PEG) is used in antibody detection and antibody identification.

“Crossmatch” refers to a method of confirming compatibility between the patient's blood (plasma) and the donor red blood cells. The crossmatch is meant primarily to detect and prevent ABO incompatibility. A serological crossmatch assay (either IS crossmatch or AHG phase crossmatch) involves the direct mixing of donor red blood cells with recipient plasma and scores for hemolysis and agglutination following immediate-spin method or AHG test.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

### Example 1: Drugs that Comprise (i) an Antibody Fc Region and (ii) a Moiety that Binds to Human CD47 Interfere with Routine Serological Assays

CD47 is a widely-expressed cell surface protein that binds to signal regulatory protein-α (SIRPα) and inhibits phagocytosis (Jaiswal et al., Trends Immunol (2010) 31(6):212-219; Brown et al., Trends Cell Biol (2001) 11(3):130-135). A wide variety of malignant tumors, including hematological and solid tumors, exhibit elevated CD47 expression, which correlates with aggressive disease (Willingham et al., Proc Natl Acad Sci USA (2012) 109(17):6662-6667). Several cancer therapies targeting CD47 have been developed to block the SIRPα-CD47 interaction, thereby permitting macrophages to carry out their phagocytic function to clear tumor cells. Importantly, because CD47 is also expressed on the surface blood cells such as of red blood cells (RBCs) and platelets (Oldenborg et al., Science (2000) 288(5473):2051-2054), CD47-binding drugs could interfere with blood typing and serological tests.

For example, CD47-targeting drugs could interfere with blood typing and serological tests by blocking and/or causing steric interference on the surface of blood cells including RBCs and platelets. In addition, anti-CD47 drugs comprising Fc regions, such as anti-CD47 antibodies, could interact with reagents used in serological tests, leading to agglutination of blood cells and false reactivity readings.

Drug A was used to determine the extent to which antibody-based drugs targeting CD47 interfere with blood typing and serological assays. Drug A is an exemplary CD47-binding drug comprising a SIRPα variant (i.e., a CD47-binding domain derived from human SIRPα) and an Fc variant derived from the Fc region of human immunoglobulin IgG1.

Materials and Methods

Detecting Interference by Drug a in Blood Typing and Serological Assays

Drug A was mixed with 5 mL of type A, B, and O donor blood samples at concentrations of 0.1 μg/mL, 0.5 μg/mL, 1 μg/mL, 5 μg/mL, or 10 μg/mL. The mixtures were incubated overnight at 2-8° C. Interference of Drug A with the following blood typing and serological tests was then determined according to the Bonfils Reference Lab procedures using the tube method:

Blood Typing Assays

Donor blood samples incubated with Drug A (as described above) were recovered and subjected to ABO and RhD typing using slide test with reagent RBCs that are known to express particular cell surface antigens and scored for agglutination.

Anti-Immunoglobulin and Complement Assays

Plasma samples were recovered from each of the donor blood samples that were incubated with Drug A. The plasma samples were then used in serological tests with reagent RBCs (Immucor). Immediate spin crossmatch (or immediate spin phase) was performed by mixing the plasma samples with reagent RBCs, immediately centrifuging the mixture, and examining the centrifuged mixture to detect agglutination. Antibody tests were also performed with the plasma samples using PEG enhancement (11.5% PEG w/v) followed by addition of anti-human globulin (AHG) reagent. Another set of antibody tests was performed with the plasma samples using PEG enhancement (11.5% PEG w/v) followed by addition of AHG reagent in order to determine the effects dithiothreitol (DTT), ficin, and/or allo-adsorption treatments on readout. Standard protocols were used for each assay, and autocontrols (i.e., wherein serum from the donor samples containing Drug A were mixed with RBCs from donor samples containing drug A) were performed in parallel.

In addition, RBC samples were recovered from each of the donor blood samples that were incubated with Drug A. The RBC samples were then used in direct antiglobulin tests (DAT), which entailed combining the RBCs with polyspecific AHG reagent (which binds to IgG and complement) or AHG reagent (which binds only to complement). In addition, antibodies bound to RBCs in each sample were eluted and tested with reagent RBCs, i.e., to detect/identify autoantibodies and/or alloantibodies. Standard protocols were used.

Results

As shown in Table 1, Drug A did not interfere with ABO/RhD typing or antibody screening with immediate spin crossmatch (IS) at any of the concentrations of Drug A that were tested (up to 10 μg/mL). However, Drug A did interfere with antibody screening with AHG reagent and PEG enhancement at the highest dose of 10 μg/mL, showing a reactivity level of 3+. The antibody screen assay with AHG reagent and PEG enhancement was also carried out with autocontrol antibodies, revealing that Drug A increased the level of reactivity in a dose-dependent manner (from weak reactivity at 0.1 μg/mL Drug A to 4+ reactivity at 10 μg/mL Drug A).

Neither DTT nor ficin resolved the reactivity caused by 10 μg/mL Drug A in the antibody screening assay with AHG reagent and PEG enhancement. By contrast, allo-adsorption testing resolved the reactivity caused by Drug A in the antibody screening assay with PEG enhancement, suggesting that Drug A interference with serological tests may be diminished or resolved by saturation binding on reagent blood cells.

DAT testing for IgG showed that Drug A caused reactivity in a dose dependent manner (from weak reactivity at 0.1 μg/mL Drug A to 3+ reactivity at 10 μg/mL Drug A). By contrast, no reactivity was observed during DAT testing for complement at any concentration of Drug A that was tested (up to 10 μg/mL), suggesting that Drug A interference is caused by IgG antibody-antigen interactions rather than complement interactions. Moderate reactivity was observed with eluate testing at all tested Drug A concentrations (reactivity of 2+ to 3+).

TABLE 1
Presence of Drug A on the Reactivity of Serological Testing

| Concentration | | | AB screen | Autocontrol | DAT | | |
|---|---|---|---|---|---|---|---|
| of spiked | | Ab | with PEG | with PEG | poly | | |
| Drug A | ABO/Rh | screen | enhancement | enhancement | and | DAT | Eluate |
| (μg/ml) | typing | via IS | at AHG | at AHG | IgG | complement | testing |
| 0.1 | NI | NI | NI | weak | weak | NI | 2+ |
| 0.5 | NI | NI | NI | 1+ | 1+ | NI | 2+ |
| 1.0 | NI | NI | NI | 3+ | 2+ to 3+ | NI | 2+ to 3+ |
| 5.0 | NI | NI | NI | 3+ | 2+ to 3+ | NI | 2+ to 3+ |
| 10.0 | NI | NI | 3+ | 4+ | 3+ | NI | 2+ to 3+ |

NI = no interference
ANG = anti-human globulin
DTT and ficin treatment did not resolve the reactivity observed with PEG enhancement
Adsorption testing resolved the reactivity observed with PEG enhancement

The results discussed above and in presented in Table 1 indicated that Drug A interferes with routine serological assays. The interference is likely due to binding of plasma Drug A to CD47 on blood cells, which is subsequently detected by IgG-specific reagents commonly used in serological testing, such as AHG.

### Example 2: Mitigation of Interference with Serological Testing Caused by Fc-Containing Proteins

The results presented in Example 1 suggested that drugs comprising (i) an antibody Fc region and (ii) a moiety that binds to human CD47 are capable of interfering with routine blood typing and serological assays. Such interference could jeopardize patient safety by causing delays in issuing of donor blood products for patients in need of transfusions. The ability of Agent B, an exemplary drug neutralizing agent that comprises a CD47 monomer, to prevent or reduce interference of Drug A in serolgoical tests was assessed as described below.

### Materials and Methods

Drug A was combined with 5 mL of donor whole blood at a concentration of 10 μg/mL and incubated overnight at about 2° C.-8° C. Plasma samples were obtained from each of Drug A-spiked donor whole blood and aliquoted. Agent B was then added to the plasma samples in a 1:9 ratio (9 drops of plasma to one drop of Agent B) using Agent B at a concentration of 2.75 mg/mL or 0.275 mg/mL. The final concentration of Agent B in each of the plasma samples was 17.5 μM or 1.75 M, respectively. The plasma sample/Agent B mixtures were incubated at room temperature for approximately 30 minutes. The serological tests described in Example 1 were then carried out on plasma samples treated with Agent B.

Results

As shown in Table 2, Agent B at a concentration of 17.5 μM mitigated the interference of Drug A (10 μg/mL) in the serological assays described in in Example 1 (see Table 1). At the lower concentration of 1.7 μM, however, Agent B did not eliminate the reactivity observed with Drug A (see Table 2).

TABLE 2
Neutralization of Drug A with CD47
Agent B in Resolution of Reactivity

| Concentration | Concentration | |
|---|---|---|
| of Drug A | of Agent B | Result |
| 10 μg/mL | 1.7 μM in | Reactivity observed in antibody |
| | a 1:9 ratio | screening with PEG enhancement. |
| 10 μg/mL | 17.5 μM in | No reactivity observed in antibody |
| | a 1:9 ratio | screening with PEG enhancement. |

Thus, the results discussed above indicated that CD47 Agent B can neutralize Drug A and mitigate its interference with serological tests.

### Example 3. Mitigating the Interference by Drugs that Comprise (i) an Antibody Fc Region and (ii) a Moiety that Binds to Human CD4 in Routine Serological Tests Using Agent B, an Exemplary Soluble CD47 Monomer or Agent C, an Exemplary Soluble a SIRPα Monomer

Introduction

CD47 is a widely expressed cell-surface protein that functions as a marker of self and provides a “don't eat me” signal by binding to signal regulatory protein-α (SIRPα), its natural receptor on macrophages, to inhibit phagocytosis (Jaiswal et al., Trends Immunol (2010) 31(6):212-219; Brown et al., Trends Cell Biol (2001) 11(3):130-135). Tumor cells overexpress CD47 to evade immune surveillance (Willingham et al., Proc Natl Acad Sci USA (2012) 109(17):6662-6667). Abundant CD47 expression has been observed on a wide variety of malignant tumors, including hematological and solid tumors, where elevated CD47 expression correlates with aggressive disease and decreased probability of survival (Willingham et al., Proc Natl Acad Sci USA (2012) 109(17):6662-6667; Chao et al. Cell. (2010) 142(4):699-713).

CD47 is widely expressed on the surface of human RBCs (Oldenborg et al., Science (2000) 288(5473):2051-2054). In the presence of circulating Drug A following dosing in patients, it is possible that Drug A from recipient patients may bind to CD47 on reagent or donor RBCs. As discussed in Examples 1 and 2, Drug A is an exemplary CD47-binding drug comprising a SIRPα variant (i.e., a CD47-binding domain derived from human SIRPα) and an Fc variant derived from the Fc region of human immunoglobulin IgG1. Since Drug A contains an Fc region of IgG1, its binding to RBC surface CD47 may resemble an antibody-antigen interaction, causing assay interference with routine blood typing and screening serological testing for pretransfusion blood bank testing. Previous in vitro study suggested that the presence of Drug A in plasma does not interfere with ABO/Rh typing and Antibody Screening with Immediate Spin. However, at a concentration of 10 μg/mL, Drug A in plasma interfered with Antibody Screening with PEG enhancement.

The experiments described below were conducted to evaluate whether the interference induced by Drug A at higher concentrations can be neutralized by the addition of CD47 monomer (i.e., Agent B described in Example 2 and below) or high affinity SIRPα monomer (i.e., Agent C, which is described in further detail below). Correlative flow cytometry analysis was conducted to evaluate if Drug A binding to RBC can be eliminated by the addition of a soluble CD47 monomer (Agent B) or a soluble high affinity SIRPα monomer (Agent C).

Materials and Methods

Agent B and Agent C were each expressed using Expi293 Expression System (Thermofisher) based on manufacturer's protocol. Both constructs contained a C-terminal His6 tag and were purified by immobilized metal affinity chromatography (IMAC). All purifications were performed using GE AktaAvant25 or Avant150. The IMAC resins used were Ni Sepharose6 Fast Flow (GE Cat No. 17-5318-01). First, the resins were equilibrated using equilibration buffer (20 mM Tris pH7, 500 mM NaCl, 5 mM Imidazole). The crude supernatant containing the his-tagged proteins was loaded through the resin. The resins were re-equilibrated with ˜20-30 column volume of equilibration buffer followed with 20-30 column volume of wash buffer (20 mM Tris pH7, 500 mM NaCl, 40 mM Imidazole). The proteins were eluted with ˜10 column volumes of elution buffer (20 mM Tris pH7, 500 mM NaCl, 250 mM Imidazole). The eluted proteins were immediately polished via gel filtration and resuspended in 1×PBS (137 mM sodium chloride, 2.7 mM potassium chloride, 4.3 mM sodium phosphate (dibasic, anhydrous), 1.4 mM potassium phosphate (monobasic, anhydrous)).

Patient serum samples used in this study were collected as a part of routine care in a hospital setting.

Anti-E is a frequently identified clinically significant alloantibody. Pooled patient serum with anti-E positive results were obtained from a hospital transfusion service for research use.

The RBC reagent cells used in this study were obtained from Biorad ID-DiaCell I-II-III.

Experimental Protocols

### Column Agglutination Tests (CATs) at Anti-Human Globulin (AHG) Phase Using BioRad Antibody Screening Cells I and II

Drug A was assessed for the potential to interfere with serological blood bank testing at concentrations up to 500 μg/mL. Drug A-spiked serum underwent antibody screening by the gel IAT (ID-Card LISS/Coombs, BIO-RAD) using RBC reagent cells ID-DiaCell I-II (Biorad). Specifically, 25 μL of Drug A-spiked serum (0.1, 1.0, 10.0, 100.0, and 500.0 μg/mL) and 50 μL of 0.8% reagent RBCs suspended in low ionic strength saline (LISS) were added to the LISS/Coombs card. After a 15 min incubation at 37° C., the card was centrifuged at 1,030 rpm for 10 min. The strength of agglutination was graded as 0 (no agglutination), 0.5+(very weak agglutination), 1+(weak agglutination), 2+(moderate agglutination), 3+(strong agglutination), or 4+(very strong agglutination).

Flow Cytometric Analysis of Drug A Binding to RBCs

Flow cytometry was performed to measure Drug A binding to reagent RBCs and reduction of the binding by using Agent B or Agent C. 25 μL Drug A-spiked serum (0.1, 1.0, 10.0, 100.0, and 500.0 μg/mL) or normal serum testing negative for unexpected antibody was added to 50 μL reagent RBCs (ID-DiaCell I). 25 μL Drug A-spiked serum (500.0 μg/mL) preincubated with 10-, 30-, and 50-fold molar excess of Agent B was added to 50 μL test RBCs (ID-DiaCell I). 25 μL Drug A-spiked serum (500.0 μg/mL) was added to 50 μL reagent RBCs (ID-DiaCell I) preincubated with 10-, 50-, 100-, 300-, and 500-fold molar excess of Agent C. The mixtures were incubated at 37° C. for 15 minutes, followed by four washes with PBS. The washed mixtures were incubated at 4° C. for 30 minutes with 0.5 μL of FITC-conjugated F(ab′)₂ fragment goat anti-human IgG (Jackson ImmunoResearch, West Grove, Pa.) and then washed twice with PBS. At least 20,000 events per sample were acquired on a FACSCanto II flow cytometer (BD Biosciences, San Jose, Calif.), and RBCs were gated by forward and side scatter parameters.

Results

Confirmation of Interference Due to Drug A

Based on previous results on Drug A's potential to interfere with RBC antibody screening (see, e.g., Example 1), Drug A was spiked into normal pooled serum that had been confirmed as red cell antibody free. Patient serum samples used in this study were collected as a part of routine care in a hospital setting. Final concentrations of Drug A were 0.1, 1, 10, 100 and 500 g/mL. The highest concentration exceeded the mean Cₘₐₓ of 247±32.5 μg/mL observed at 10 mg/kg QW dose level observed in human patients (see Jin et al. “Pharmacokinetic and Pharmacodynamic Characterization of DRUG A, a CD47 Blocker, in Patients with Advanced Malignancy and Non-Hodgkin Lymphoma.” (Poster) Society for Immunotherapy of Cancer Annual Meeting, Washington, D.C., Nov. 7-11, 2018).

Column agglutination tests (CATs) at anti-human globulin (AHG) phase using BioRad antibody screening cells I and II were performed. The strength of reaction ranged from 2+ to 3+ on a scale of 0 to 4+ were observed across the Drug A concentrations tested, suggesting interference due to the binding of Drug A to RBCs and the interaction of the Fc portion of Drug A with the AHG reagent (Table 3).

TABLE 3
Drug A-Spiked Serum Tested Positive In Column Agglutination
Tests at Anti-Human Globulin (AHG) Phase

| Spiked Drug A concentration | I cell | II cell |
|---|---|---|
| (μg/mL) | (0.8% RBC) | (0.8% RBC) |
| 500 | +3 | +3 |
| 100 | +3 | +3 |
| 10 | +3 | +3 |
| 1 | +2 | +2 |
| 0.1 | +2 | +2 |

Mitigation of Interference Using Agent B

Agent B is a CD47 monomer that comprises the IgSF domain. It was postulated that the binding of Agent B to Drug A in solution would form an Agent B-Drug A complex. It was also postulated that the Agent B-Drug A complex would be unable to bind to endogenous CD47 expressed on RBCs, thus preventing Drug A from interfering with RBC antibody screening tests. To test this possibility, Agent B, at concentrations corresponding to 10× to 500× molar ratio to Drug A, was added to Drug A-spiked serum. After a 30 minute incubation at room temperature (RT), CATs at AHG phase using BioRad antibody screening cells I and II were again performed. Resolution of Drug A interference was achieved at 40× to 100× molar ratios of Agent B (Table 4).

TABLE 4
Agent B Mitigates Interference Due to Drug A In Column
Agglutination Tests at Anti-Human Globulin (AHG) Phase

| | X10 | | X20 | | X30 | | X40 | |
|---|---|---|---|---|---|---|---|---|
| Spiked | I cell | II cell | I cell | II cell | I cell | II cell | I cell | II cell |
| Drug A | (0.8% | (0.8% | (0.8% | (0.8% | (0.8% | (0.8% | (0.8% | (0.8% |
| (μg/mL) | RBC) | RBC) | RBC) | RBC) | RBC) | RBC) | RBC) | RBC) |
| 500 | +1 | +1 | +/− | +/− | −/+ | −/+ | − | − |
| 100 | +1 | +1 | +1 | +/− | 1+ | −/+ | − | − |
| 10 | +1 | +1 | +1 | +1 | +1 | −/+ | − | − |
| 1 | +2 | +2 | +1 | +1 | +1 | +1 | +1 | +1 |
| 0.1 | +1 | +1 | +1 | +1 | +1 | +1 | +1 | +1 |
| | X50 | | X100 | | X200 | | X300 | |
| Spiked | I cell | II cell | I cell | II cell | I cell | II cell | II cell | I cell |
| Drug A | (0.8% | (0.8% | (0.8% | (0.8% | (0.8% | (0.8% | (0.8% | (0.8% |
| (μg/mL) | RBC) | RBC) | RBC) | RBC) | RBC) | RBC) | RBC) | RBC) |
| 500 | − | − | NT* | NT* | NT* | NT* | NT* | NT* |
| 100 | − | − | NT* | NT* | NT* | NT* | NT* | NT* |
| 10 | − | − | NT* | NT* | NT* | NT* | NT* | NT* |
| 1 | +/− | +/− | − | − | − | − | − | − |
| 0.1 | −/+ | +/− | − | − | − | − | − | − |

| | X50 | |
|---|---|---|
| Spiked | I cell | I cell |
| Drug A | (0.8% | (0.8% |
| (μg/mL) | RBC) | RBC) |
| 500 | NT* | NT* |
| 100 | NT* | NT* |
| 10 | NT* | NT* |
| 1 | NT* | NT* |
| 0.1 | NT* | NT* |

*NT: not tested

209.82 μM Agent B (3.32 mg/mL) was used for neutralization. Specifically, 25 μL Drug A-spiked plasma (500 μg/mL, 160 pmol) was incubated with 8, 16, 24, 32, and 40 μL Agent B (10×, 1.6 nmol; 20×, 3.2 nmol; 30×, 4.8 nmol; 40×, 6.4 nmol; 50×, 8.0 nmol). 25 μL Drug A-spiked plasma (100 μg/mL, 32 pmol) was incubated with 1.6, 3.2, 4.8, 6.4, and 8.0 μL Agent B (10×, 320 pmol; 20×, 640 pmol; 30×, 960 pmol; 40×, 1.28 nmol; 50×, 1.6 nmol). 25 μL Drug A-spiked plasma (10 μg/mL, 3.2 pmol) was incubated with 160, 320, 480, 640, and 800 nL Agent B (10×, 32 pmol; 20×, 64 pmol; 30×, 96 pmol; 40×, 128 pmol; 50×, 160 pmol). 25 μL Drug A-spiked plasma (1 μg/mL, 320 fmol) was incubated with 16, 32, 48, 64, 80, 160, 320, and 480 nL Agent B (10×, 3.2 pmol; 20×, 6.4 pmol; 30×, 9.6 pmol; 40×, 12.8 pmol; 50×, 16.0 pmol; 100×, 32.0 pmol; 200×, 64.0 pmol; 300×, 96.0 pmol). 25 μL Drug A-spiked plasma (0.1 μg/mL, 32 fmol) was incubated with 1.6, 3.2, 4.8, 6.4, 8.0, 16.0, 32.0, and 48.0 nL Agent B (10×, 320 fmol; 20×, 640 pmol; 30×, 960 fmol; 40×, 1.28 pmol; 50×, 1.60 pmol; 100×, 3.2 pmol; 200×, 6.4 pmol; 300×, 9.6 pmol).

Mitigation of Interference Using Agent C

Agent C is a recombinant high affinity SIRPα monomer (Kd for human CD47=14.4 μM). It was postulated that the binding of Agent to CD47 expressed on RBCs would block Drug A from binding the CD47 expressed on RBCs, thus preventing of Drug A from interfering with RBC antibody screening tests. To test this possibility, BioRad antibody screening cells I and II were incubated with Agent C at concentrations corresponding to 10× to 500× molar ratio to Drug A. After a 30 minute incubation at room temperature (RT), Agent C-incubated reagent RBCs were tested in CATs at AHG phase with Drug A-spiked serum. Resolution of Drug A interference was achieved at 300× molar ratios of Agent C (Table 5).

TABLE 5
Agent C Mitigates Interference Due to Drug A in Column
Agglutination Tests at Anti-Human Globulin (AHG) Phase

| | X10 | | X20 | | X30 | | X40 | |
|---|---|---|---|---|---|---|---|---|
| Spiked | I cell | II cell | I cell | II cell | I cell | II cell | I cell | II cell |
| Drug A | (0.8% | (0.8% | (0.8% | (0.8% | (0.8% | (0.8% | (0.8% | (0.8% |
| (μg/mL) | RBC) | RBC) | RBC) | RBC) | RBC) | RBC) | RBC) | RBC) |
| 500 | +2 | +2 | +2 | +2 | +2 | +2 | +2 | +2 |
| 100 | +1 | +1 | +1 | +1 | +1 | +1 | +1 | +1 |
| 10 | +2 | +2 | +2 | +2 | +2 | +2 | +2 | +2 |
| 1 | +2 | +2 | +2 | +2 | +2 | +2 | +2 | +2 |
| 0.1 | +2 | +2 | +2 | +2 | +2 | +2 | +2 | +2 |
| | X50 | | X100 | | X200 | | X300 | |
| Spiked | I cell | II cell | I cell | II cell | I cell | II cell | I cell | II cell |
| Drug A | (0.8% | (0.8% | (0.8% | (0.8% | (0.8% | (0.8% | (0.8% | (0.8% |
| (μg/mL) | RBC) | RBC) | RBC) | RBC) | RBC) | RBC) | RBC) | RBC) |
| 500 | +1 | +1 | +1 | +/− | +1 | +1 | +1 | +/− |
| 100 | +1 | +1 | +1 | +1 | +1 | +1 | +1 | +1 |
| 10 | +1 | +1 | +1 | +1 | +1 | +1 | +1 | +1 |
| 1 | +1 | +1 | +1 | +1 | +1 | +1 | +1 | +1 |
| 0.1 | +2 | +2 | +1 | +1 | +2 | +2 | +1 | +1 |

| | X500 | |
|---|---|---|
| Spiked | I cell | I cell |
| Drug A | (0.8% | (0.8% |
| (μg/mL) | RBC) | RBC) |
| 500 | NT* | NT* |
| 100 | − | − |
| 10 | − | − |
| 1 | − | − |
| 0.1 | − | − |

NT: not tested

345.66 M Agent C (4.77 mg/mL) was used for masking the Drug A binding site on CD47 of reagent RBCs. Specifically, 25 μL Drug A-spiked plasma (500 μg/mL, 160 pmol) was incubated with 5, 10, 15, 20, 25, 50, and 150 μL Agent C (10×, 1.6 nmol; 20×, 3.2 nmol; 30×, 4.8 nmol; 40×, 6.4 nmol; 50×, 8.0 nmol; 100×, 16.0 nmol; 300×, 48.0 nmol). 25 μL Drug A-spiked plasma (100 g/mL, 32 pmol) was incubated with 1, 2, 3, 4, 5, 10, 30, and 50 μL Agent C (10×, 320 pmol; 20×, 640 pmol; 30×, 960 pmol; 40×, 1.28 nmol; 50×, 1.6 nmol; 100×. 3.2 nmol; 300×, 9.6 nmol; 500×, 16 nmol). 25 μL Drug A-spiked plasma (10 μg/mL, 3.2 pmol) was incubated with 0.1, 0.2, 0.3, 0.4, 0.5, 1.0, 3.0, and 5.0 μL Agent C (10×, 32 pmol; 20×, 64 pmol; 30×, 96 pmol; 40×, 128 pmol; 50×, 160 pmol, 100×, 320 pmol; 300×, 960 pmol; 500×, 1.6 nmol). 25 μL Drug A-spiked plasma (1 μg/mL, 320 fmol) was incubated with 10, 20, 30, 40, 50, 100, 300, and 500 nL Agent C (10×, 3.2 pmol; 20×, 6.4 pmol; 30×, 9.6 pmol; 40×, 12.8 pmol; 50×, 16.0 pmol; 100×, 32.0 pmol; 300×, 96.0 pmol; 500×, 160.0 pmol). 25 μL Drug A-spiked plasma (0.1 μg/mL, 32 fmol) was incubated with 1, 2, 3, 4, 5, 10, 30, and 50 nL Agent C (10×, 320 fmol; 20×, 640 pmol; 30×, 960 fmol; 40×, 1.28 pmol; 50×, 1.60 pmol; 100×, 3.2 pmol; 300×, 9.6 pmol; 500×, 16.0 pmol).

### Mitigation of Interference Using Either Agent B or Agent C Allows Detection of RBC Alloantibody in Patient Serum

To investigate whether mitigation of Drug A interference by Agent B or Agent C would allow detection of a true RBC alloantibody, pooled human serum containing anti-E, an exemplary an RBC alloantibody, was used in experiments similar to those described above. Anti-E is a frequently identified clinically significant alloantibody and pooled patient serum with anti-E positive results were obtained from a hospital transfusion service for research use. Patient serum samples used in this study were collected as a part of routine care. Patient serum containing anti-E was spiked with Drug A at 0.1 to 500 μg/mL. A CAT at AHG phase was performed with Biorad I and II cells. While Biorad II cells are E-positive, Pan-positivity was observed with both reagent cells at all Drug A concentrations (Table 6A). However, following incubations with 32 μL Agent B (at 3.32 mg/mL), which is 40× the molar ratio of 25 μL Drug A (500 μg/ml) for 30 min at RT, the CAT at AHG phase only revealed positivity with Biorad II cells, suggesting that only anti-E activity was detected and Drug A interference was resolved at all Drug A concentrations tested (Table 6B).

Similar results were observed with Agent C. BioRad antibody screening cells I and II were incubated with 150 μL Agent C (at 4.77 mg/mL), which is a 300× molar ratio of 25 μL Drug A (500 μg/ml) with for 30 min at RT. CATs at AHG phase with the Drug A-spiked patient serum containing anti-E were performed and only reveal positivity with Biorad II cells, suggesting that only anti-E activity was detected and Drug A interference was resolved at all Drug A concentrations tested (Table 6C).

### Tables 6A-6C. Mitigation Interference Using Either Agent B or Agent C Allows Detection of RBC Alloantibody in Patient Serum

TABLE 6A

| Spiked Drug A | I cell | II cell |
|---|---|---|
| (μg/mL) | (0.8% RBC) | (0.8% RBC)* |
| 500 | +3 | +4 |
| 100 | +3 | +4 |
| 10 | +3 | +4 |
| 1 | +2 | +4 |
| 0.1 | +2 | +4 |

*Biorad II cell is E-positive.

Table 6B^{‡}

X40 Agent B (6.4 nmol)

| Spiked Drug A | I cell | II cell |
|---|---|---|
| (μg/mL) | (0.8% RBC) | (0.8% RBC)* |
| 500 | — | +4 |
| 100 | — | +4 |
| 10 | — | +4 |
| 1 | — | +4 |
| 0.1 | — | +4 |

*Biorad II cell is E-positive.
^{‡}6.4 nmol Agent B (40x molar ratio of 500 μg/mL Drug A) was used for all Drug A concentrations tested.

Table 6C^{‡}

X300 Agent C (48.0 nmol)

| Spiked Drug A | I cell | II cell |
|---|---|---|
| (μg/mL) | (0.8% RBC) | (0.8% RBC)* |
| 500 | — | +4 |
| 100 | — | +4 |
| 10 | — | +4 |
| 1 | — | +4 |
| 0.1 | — | +4 |

*Biorad II cell is E-positive.
^{‡}48 nmol Agent C (300x molar ratio of 500 μg/mL Drug A) was used for all Drug A concentrations tested.

Correlative Analysis of Drug a Binding to RBCs by Flow Cytometry

To determine whether the binding of Drug A to CD47 expressed on reagent RBCs is actually reduced by the presence of a soluble high affinity SIRPα (Agent C) or soluble CD47 (Agent B), flow cytometric analyses were performed as follows: Drug A was spiked into normal serum at 0 (negative control), 0.1, 1.0, 10, 100, and 500 μg/mL. BioRAD I cells were incubated with the Drug A-spiked serum samples for 15 minutes at 37° C. FITC-conjugated anti-human IgG was used for staining Drug A-bound RBCs. Cells were analyzed by flow cytometry and MFI (mean fluorescence intensity) was determined.

To evaluate the impact of Agent B on the binding of Drug A to RBCs, Drug A-spiked serum samples, and a non-spiked negative control sample, were incubated with Agent B (concentration ranging from 10× to 50× molar ratio of Drug A) at room temperature for 15 minutes. BioRAD I cells were incubated with the serum samples for 15 minutes at 37° C. and subjected to flow cytometric analysis.

To evaluate the impact of Agent C on the binding of Drug A to RBCs, Agent C (concentrations ranging from 10× to 500× molar ratio of Drug A) was added to BioRAD I cells for 15 minutes at 37° C. The cells were further incubated with Drug A-spiked serum samples, as well as a non-spiked negative control sample, for 15 minutes at 37° C. and subjected to flow cytometric analysis.

RBCs were bound with Drug A contained in serum as indicated by increased FITC signal. A concentration-dependent increase in MFI was observed with increasing concentration of Drug A being spiked into the serum (Table 7A). Both Agent B and Agent C=reduce Drug A binding to RBCs effectively and in a concentration-dependent manner (Tables 7B and 7C).

### Tables 7A-7C. Agent B and Agent C Reduce the Binding of Drug A to RBCs

TABLE 7A

| | | MFI (FITC |
|---|---|---|
| | Concentration | Channel) |
| Drug A | 0 | 8 |
| | 0.1 | 403 |
| | 1 | 2941 |
| | 10 | 3308 |
| | 100 | 4084 |
| | 500 | 4858 |

TABLE 7B

| Drug A | | | | |
|---|---|---|---|---|
| Concentration | Agent B | Agent B | | MFI (FITC |
| (μg/ml) | Molar Ratio | mols | | Channel) |
| 0.1 | 10x | 0.32 | pmol | 125 |
| | 30x | 0.96 | nmol | 105 |
| | 50x | 1.6 | pmol | 77 |
| 1 | 10x | 3.2 | pmol | 212 |
| | 30x | 9.6 | pmol | 52 |
| | 50x | 16.0 | pmol | 26 |
| 10 | 10x | 32 | pmol | 184 |
| | 30x | 96 | pmol | 99 |
| | 50x | 160 | pmol | 41 |
| 100 | 10x | 320 | pmol | 90 |
| | 30x | 960 | pmol | 28 |
| | 50x | 1600 | pmol | 22 |
| 500 | 10x | 1.6 | nmol | 67 |
| | 30x | 4.8 | nmol | 71 |
| | 50x | 8.0 | nmol | 40 |

TABLE 7C

| Drug A | | | | |
|---|---|---|---|---|
| Concentration | Agent C | Agent C | | MFI (FITC |
| (μg/ml) | Molar Ratio | mols | | Channel) |
| 0.1 | 10x | 0.32 | pmol | 62 |
| | 50x | 1.6 | pmol | 40 |
| | 100x | 3.2 | pmol | 28 |
| | 300x | 9.6 | pmol | 16 |
| | 500x | 16.0 | pmol | 7 |
| 1 | 10x | 3.2 | pmol | 87 |
| | 50x | 16.0 | pmol | 41 |
| | 100x | 32.0 | pmol | 15 |
| | 300x | 96.0 | pmol | 16 |
| | 500x | 160 | pmol | 10 |
| 10 | 10x | 32 | pmol | 138 |
| | 50x | 160 | pmol | 8 |
| | 100x | 320 | pmol | 47 |
| | 300x | 960 | pmol | 15 |
| | 500x | 1.6 | nmol | 15 |
| 100 | 10x | 320 | pmol | 111 |
| | 50x | 1.6 | nmol | 20 |
| | 100x | 3.2 | nmol | 35 |
| | 300x | 9.6 | nmol | 31 |
| | 500x | 16.0 | nmol | 19 |
| 500 | 10x | 1.6 | nmol | 89 |
| | 50x | 8.0 | nmol | 42 |
| | 100x | 16.0 | nmol | 50 |
| | 300x | 48.0 | nmol | 40 |
| | 500x | 80.0 | nmol | 42 |

Conclusions

In column agglutination tests (CATs) at anti-human globulin (AHG) phase using BioRad antibody screening cells I and II, the presence of Drug A in serum at concentrations up to 500 μg/mL interfered with the test and produced false positive results. Using a recombinant CD47 monomer (Agent B) or a high affinity SIRPα monomer (Agent C), this interference was resolved. In addition, anti-E, a clinically relevant alloantibody, was detectable in serum after resolution of interference by Drug A with either Agent B or Agent C. Correlative flow cytometric analysis suggested that Drug A binds to RBC in a concentration-dependent manner. Both Agent B and Agent C reduced Drug A binding to RBCs, consistent with the neutralization of the interference of Drug A that binds CD47 expressed on RBC.

### REFERENCES

Brown E J, Frazier W A. Integrin-associated protein (CD47) and its ligands. Trends Cell Biol. 2001 March; 11(3):130-5.
Chao M P, Alizadeh A A, Tang C, Myklebust J H, Varghese B, Gill S, et al. Anti-CD47 antibody synergizes with rituximab to promote phagocytosis and eradicate non-Hodgkin lymphoma. Cell. 2010 Sep. 3; 142(4):699-713.
Jaiswal S, Chao M P, Mejeti R and Weissman I L. Macrophages as mediators of tumor immunosurveillance. Trends Immunol. 2010 June; 31(6): 212-219.
Oldenborg P A, Zheleznyak A, Fang Y F, Lagenaur C F, Gresham H D et al. Role of CD47 as a marker of self on red blood cells. Science 2000 Jun. 16; 288 (5473):2051-4.
Weiskopf K, Ring A M, Ho C C, Volkmer J-P, Levin A M, Volkmer A K, et al. Engineered SIRPα variants as immunotherapeutic adjuvants to anticancer antibodies. Science. 2013 Jul. 5; 341(6141):88-91.
Willingham S B, Volkmer J P, Gentles A J, Sahoo D, Dalerba P, Mitra S S, et al. The CD47-signal regulatory protein alpha (SIRPα) interaction is a therapeutic target for human solid tumors. Proc Natl Acad Sci USA. 2012 Apr. 24; 109(17):6662-7.
Zhao X W, Van Beek E M, Schornagela K, Van der Maadenb H, Houdta M V; CD47-signal regulatory protein-α (SIRPα) interactions form a barrier for antibody-mediated tumor cell destruction. Proc Natl Acad Sci USA. 2011 Nov. 8; 108(45):18342-7.
Technical Manual. Current Edition. Bethesda: AABB.

Summary

In column agglutination tests (CATs) at anti-human globulin (AHG) phase using BioRad antibody screening cells I and II, the presence of Drug A in serum showed interference with the test and caused false positive results. Using a recombinant CD47 monomer (Agent B) or a high affinity SIRPα monomer (Agent C), this interference was resolved. In addition, anti-E, a clinically relevant alloantibody, remained to be detected, after resolution of interference by either Agent B or Agent C. Correlative flow cytometric analysis suggested that Drug A binds to RBC in a concentration-dependent manner. Both Agent B and Agent C reduced the binding of Drug A to RBCs, consistent with the neutralization of ALX interference.

### Example 4. Mitigating the Interference by Drugs that Comprise (i) an Antibody Fc Region and (ii) a Moiety that Binds to Human CD4 in Routine Serological Tests Using an Anti-SIRPα Antibody (or Antigen-Binding Fragment Thereof)

The experiments described below were conducted to evaluate whether the interference induced by Drug A at higher concentrations can be neutralized by the addition of an anti-SIRPα antibody (or a Fab fragment thereof). Correlative flow cytometry analysis was conducted to evaluate whether Drug A bound to the CD47 on the surface of RBCs can be displaced by the addition of an anti-SIRPα antibody (or a Fab fragment thereof.)

Hemagglutination assays were performed as follows: Pooled human erythrocytes in a 3-3.4% modified Alsevers solution (Bio-Rad) were added at 40 μl per well in a 96 well plate (Falcon). Wells were incubated with either PBS (Gibco) or Drug A at 250 ng/mL for 1 hour at 37° C. and subsequently washed three times in PBS with thorough decanting after each wash. Fab fragments of Antibodies A and C, i.e., exemplary anti-SIRPα antibodies that block the interaction between SIRPα and CD47, were titrated 1:2 starting at 100 μg/mL or 625 μg/mL and incubated with the Drug A-coated erythrocytes for 1 hour at 37° C. followed by three washes with PBS and thoroughly decanting remaining liquid. Two drops of Anti-human globulin anti-IgG (Bio-Rad) were then added to each well and spun at 800 G for thirty seconds before gently dislodging pellet and jpeg image capture. Antibody A comprises a heavy chain variable domain (V_{H}) that comprises SEQ ID NO: 6 and a light chain variable domain (V_{L}) that comprises SEQ ID NO: 7. Antibody C comprises a heavy chain variable domain (V_{H}) that comprises SEQ ID NO: 21 and a light chain variable domain (V_{L}) that comprises SEQ ID NO: 22. Parallel experiments were performed with Antibody B, i.e., an exemplary anti-SIRPα antibody that does not block the interaction between SIRPα and CD47. Antibody B comprises a heavy chain variable domain (V_{H}) that comprises SEQ ID NO: 23 and a light chain variable domain (V_{L}) that comprises SEQ ID NO: 24. Each of Antibodies A, B, and C cross-react with SIRPβ and SIRPγ. Erythrocytes incubated with PBS or Drug A alone served as negative and positive controls for the assay, respectively.

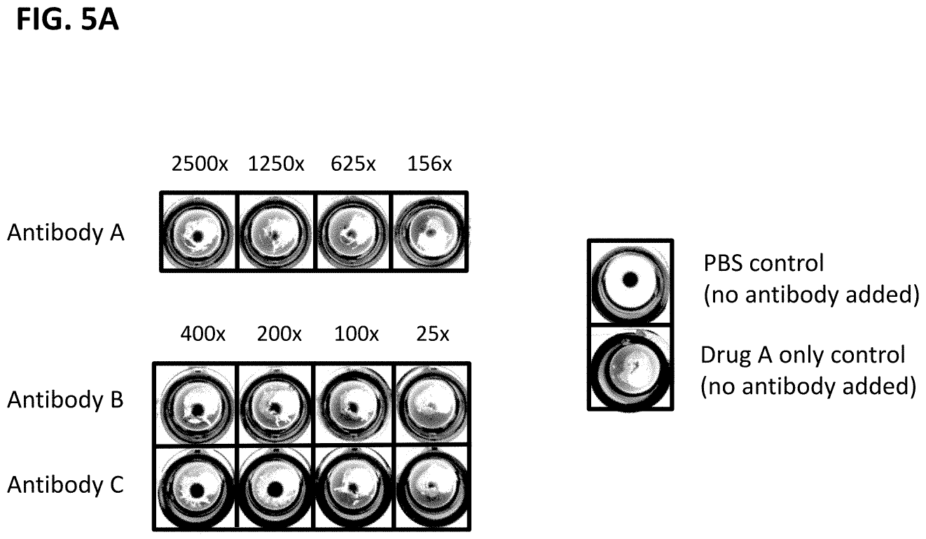

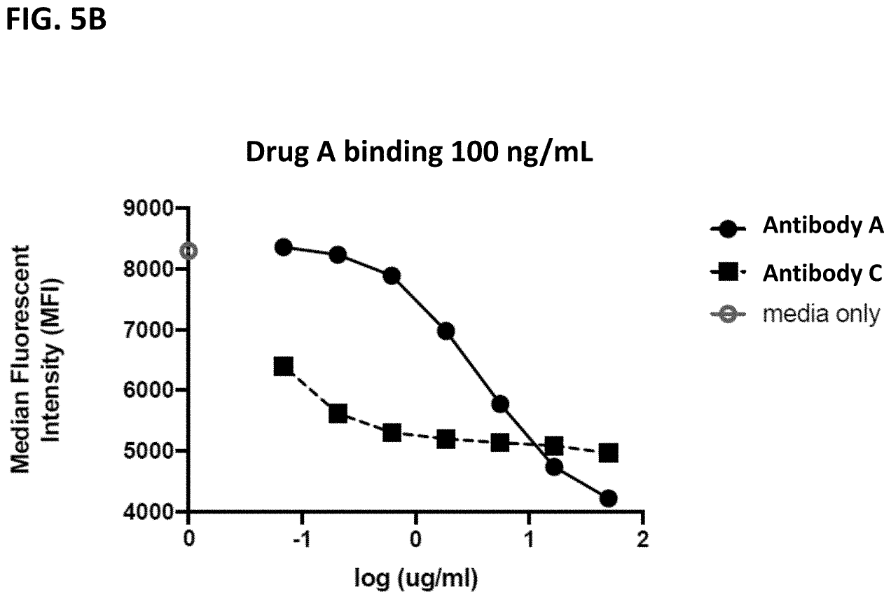

As shown in FIG. 5A, Antibody C, which blocks the interaction between SIRPα and CD47, prevented hemagglutination when added to Drug A-coated erythrocytes at a 200-fold and at a 400-fold molar excess relative to the amount of Drug A. Antibody A, which also blocks the interaction between SIRPα and CD47, prevented hemagglutination when added to Drug A-coated erythrocytes at a 2500-fold molar excess relative to the amount of Drug A. The addition of Antibody B, an anti SIRPα antibody which does not block the interaction between SIRPα and CD47, prevented hemagglutination when added to Drug A-coated erythrocytes at a 400-fold molar excess relative to the amount of Drug A. The results shown in FIG. 5A indicate that Fabs of Antibody A, Antibody B, and Antibody C each prevented hemagglutination by displacing Drug A from CD47 erythrocytes. CD47 on the surface of the erythrocytes.

To confirm that Fabs of Antibody A and Antibody C are capable of displacing Drug A bound to CD47 on the surface of erythrocytes, flow cytometry experiments were performed as follows: Drug A was fluorescently labeled with the Alexa Fluor 647 Protein Labeling Kit (Thermo Fisher Scientific) according to the manufacturer's instructions. DLD-1 human colon epithelial cells were washed once in staining buffer (PBS, 2% FBS) and stained in PBS with fixable live/dead stain (Invitrogen) for 1 hour at 4° C. Cells were then washed in staining buffer and co-incubated in a 96 well plate (Falcon) with labeled Drug A at 100 ng/mL and either unlabeled Antibody A Fab or unlabeled Antibody C Fab at a 1:2 titration starting at 50 g/mL. After a 1 hour incubation at 4° C., cells were washed twice in staining buffer and fixed in 0.5% paraformaldehyde. Cells were analyzed on an Attune (ThermoScientific), and subsequent data analysis using Flowjo 10.6. Drug A binding to CD47 on the surface of DLD-1 cells was indicated by median fluorescence intensity of Alexa Fluor 647. As shown in FIG. 5B, the addition of Antibody A and Antibody C to the Drug A-bound cells decreased Drug A binding to CD47 on the surface of DLD-1 cells. These results indicated that Antibody A and Antibody C are capable of displacing Drug A bound to CD47 on the surface of DLD-1 cells.

Taken together the results in this Example show that Antibody A, B and C can prevent interference in a routine serological assay by displacing Drug A bound to CD47 on the surface of blood cells. These results also indicate that an antibody that cross-reacts with SIRPα, SIRPβ, and SIRPγ can prevent interference in a routine serological assay by displacing Drug A bound to CD47 on the surface of blood cells.

Each embodiment herein described may be combined with any other embodiment or embodiments unless clearly indicated to the contrary. In particular, any feature or embodiment indicated as being preferred or advantageous may be combined with any other feature or features or embodiment or embodiments indicated as being preferred or advantageous, unless clearly indicated to the contrary.

All references cited in this application are expressly incorporated by reference herein.

1: A method of reducing drug interference in a serological assay using reagent red blood cells (RBC) or reagent platelets, said method comprising:
(a) adding a drug neutralizing agent that binds to a drug and blocks the drug from binding the reagent RBC or the reagent platelets to a plasma sample from a subject who has received treatment with the drug; and
(b) performing the serological assay of the plasma sample after step (a), using the reagent RBC or the reagent platelets,
wherein the drug comprises (i) a human antibody Fc region or variant thereof and (ii) a moiety that binds to human CD47.

2: The method of claim 1, wherein the moiety of the drug that binds to human CD47 comprises a wild type SIRPα, a SIRPα variant, or a fragment of the wild type SIRPα or the SIRPα variant.

3-4. (canceled)

5: The method of claim 1, wherein the drug neutralizing agent is an anti-SIRPα antibody that is capable of binding the wild type SIRPα, the SIRPα variant, or the fragment of the wild type SIRPα or the SIRPα variant.

6: The method of claim 1, wherein the moiety of the drug that binds to human CD47 comprises a wild type SIRPγ, a SIRPγ variant, or a fragment of the wild type SIRPγ or the SIRPγ variant.

7-8. (canceled)

9: The method of claim 6, wherein the drug neutralizing agent is an anti-SIRPγ antibody that is capable of binding the wild type SIRPγ, the SIRPγ variant, or the fragment of the wild type SIRPγ or the SIRPγ variant.

10: The method of claim 1, wherein the moiety of the drug that binds to human CD47 comprises a SIRPβ variant or a fragment of the SIRPβ variant.

11-12. (canceled)

13: The method of claim 10, wherein the drug neutralizing agent is an anti-SIRPβ antibody that is capable of binding the SIRPβ variant or the fragment of the SIRPβ variant.

14: The method of claim 1, wherein the drug is an anti-CD47 antibody.

15: The method of claim 14, wherein the drug neutralizing agent is an anti-idiotypic antibody that binds the antigen binding portion of the anti-CD47 antibody.

16: The method of claim 1, wherein the drug neutralizing agent is a CD47 polypeptide capable of binding the moiety of the drug that binds to human CD47.

17: The method of claim 16, wherein the CD47 polypeptide that is capable of binding the moiety of the drug that binds to human CD47:
(a) is a monomer, a dimer, or an oligomer;
(b) is a human CD47, a mouse CD47, a rat CD47, a rhesus CD47, or a cynomolgus CD47;
(c) comprises the amino acid sequence of SEQ ID NO: 1; or
(d) is a CD47 variant that comprises one or more amino acid substitutions, insertions, deletions, N-terminal extensions, or C-terminal extensions relative to the wildtype CD47.

18-20. (canceled)

21: The method of claim 17, wherein the CD47 polypeptide is CD47 variants that comprises one or more amino acid substitutions, insertions, deletions, N-terminal extensions, or C-terminal extensions relative to the wildtype CD47, and wherein the CD47 variant comprises the amino acid sequence set forth in any one of SEQ ID NOs: 2-5.

22: The method of claim 1, wherein the affinity of the drug neutralizing agent for the drug is higher than the affinity of the drug for human CD47.

23: The method of claim 1, wherein the drug neutralizing agent is added to the plasma sample in a molar excess amount relative to the amount of drug in the plasma sample.

24: A method of reducing drug interference in a serological assay of a blood sample containing red blood cells (RBC) and/or platelets, said method comprising:
(a) adding an anti-SIRPα antibody to the blood sample from a subject who has received treatment with a drug; and
(b) performing the serological assay of the blood sample after step (a),
wherein the drug comprises (i) an antibody Fc region and (ii) an extracellular domain of a wild type SIRPα or a variant thereof that binds to human CD47, and
wherein the anti-SIRPα antibody fragment displaces the drug bound to CD47 on the surface of the RBC in the blood sample.

25: The method of claim 24, wherein the anti-SIRPα antibody comprises:
(a) a heavy chain variable domain (V_{H}) that comprises SEQ ID NO: 6 and a light chain variable domain (VL) that comprises SEQ ID NO: 7;
(b) a heavy chain variable domain (V_{H}) that comprises SEQ ID NO: 21 and a light chain variable domain (VL) that comprises SEQ ID NO: 22; or
(c) a heavy chain variable domain (V_{H}) that comprises SEQ ID NO 23 and a light chain variable domain (VL) that comprises SEQ ID NO: 24;

26: The method of claim 24, wherein the drug comprises a variant of an extracellular domain of the wild type SIRPα.

27. (canceled)

28: The method of claim 24, wherein the anti-SIRPα antibody is added to the blood sample in a molar excess amount relative to the amount of drug in the blood sample.

29: A method of reducing drug interference in a serological assay using reagent red blood cells (RBCs), reagent platelets, or a combination thereof said method comprising:
(a) adding a cell binding agent to the reagent red blood cells (RBCs), reagent platelets, or combination thereof, wherein the cell binding agent binds to human CD47 and does not comprise an antibody Fc region; and
(b) performing the serological assay of a plasma sample using the reagent red blood cells (RBCs), reagent platelets, or combination thereof of step (a),
wherein the plasma sample is from a subject who has received treatment with a drug, and
wherein the drug comprises (i) an antibody Fc region and (ii) a moiety that binds to human CD47.

30: A method of reducing drug interference in a serological assay using reagent red blood cells (RBCs), reagent platelets, or a combination thereof, said method comprising:
(a) adding a cell binding agent a plasma sample from a subject who has received treatment with a drug, wherein the cell binding agent binds to human CD47 and does not comprise an antibody Fc region; and
(b) performing the serological assay of the plasma sample after step (a) using the reagent red blood cells (RBCs), reagent platelets, or combination thereof,
wherein the drug comprises (i) an antibody Fc region and (ii) a moiety that binds to human CD47.

31: A method of reducing drug interference in a serological assay of a blood sample containing reagent red blood cells (RBCs), reagent platelets, or a combination thereof, said method comprising:
(a) adding a cell binding agent that binds to human CD47 and does not comprise an antibody Fc region to a blood sample from a subject who has received treatment with a drug; and
(b) performing the serological assay of the blood sample after step (a),
wherein the drug comprises (i) an antibody Fc region and (ii) a moiety that binds to human CD47.

32: The method of claim 29, wherein the cell binding agent comprises
(a) a wild type SIRPα, wild type SIRPγ, or a fragment of the wild type SIRPα or the wild type SIRPγ that is capable of binding human CD47;
(b) a SIRPα variant that is capable of binding human CD47, or a CD47-binding fragment thereof;
(c) a SIRPβ variant that is capable of binding human CD47, or a CD47-binding fragment thereof;
(d) a SIRPγ variant that is capable of binding human CD47, or a CD47-binding fragment thereof; or
(e) an antigen-binding fragment of an anti-CD47 antibody.

33-38. (canceled)

39: The method of claim 32, wherein the cell binding agent is a SIRPα variant, a SIRPβ variant, or a SIRPγ variant that comprises the amino acid sequence of any one of SEQ ID NOs: 8-16.

40. (canceled)

41: The method of claim 32, wherein the cell binding agent comprises an antigen binding fragment of an anti-CD47 antibody, and wherein the antigen binding fragment is a Fab, a Fab′, a Fab′-SH, an F(ab′)2, an Fv, an ScFv, or a diabody.

42: The method of claim 29, wherein the affinity of the cell binding agent for human CD47 is higher than the affinity of the drug for human CD47.

43: The method of claim 29, wherein the cell binding agent is added to the reagent RBC and/or reagent platelets in a molar excess amount relative to the amount of drug in the plasma sample.

44: The method of claim 30, wherein the cell binding agent is added to the plasma sample in a molar excess amount relative to the amount of drug in the plasma sample.

45: The method of claim 31, wherein the SIRPα agent is added to the blood sample in a molar excess amount relative to the amount of drug in the blood sample.

46: The method of claim 1, wherein the antibody Fc region of the drug is a human IgG Fc region or a variant thereof.

47: The method of claim 46, wherein the human IgG Fc region is an IgG1, IgG2, or IgG4 Fc region, or a variant of an IgG1, IgG2, or IgG4 Fc region.

48: The method of claim 1, wherein the serological assay is:
(a) an ABO/Rh typing assay;
(b) an immediate spin (IS) assay;
(c) a direct antiglobulin (DAT) assay using a polyspecific reagent that detects IgG and complement C3;
(d) a direct antiglobulin (DAT) assay using a monospecific reagent that detects complement C3; or
(e) a PEG-enhanced serological assay.

49-52. (canceled)

53: The method of claim 48, wherein the serological assay is a DAT assay, and wherein an eluate test is performed following the DAT assay.

54: A polypeptide comprising any one of SEQ ID NOs: 11-24.
